SEQUENCE LISTING (17 sequences)

SEQ ID NO: 1 - DNA, 19 bp, Artificial Sequence
  F3 primer
tggctactaccgaagagct

SEQ ID NO: 2 - DNA, 20 bp, Artificial Sequence
  B3 primer
tgcagcattgttagcaggat

SEQ ID NO: 3 - DNA, 41 bp, Artificial Sequence
  FIP primer
tctggcccagttcctaggtagtccagacgaattcgtggtgg

SEQ ID NO: 4 - DNA, 40 bp, Artificial Sequence
  BIP primer
agacggcatcatatgggttgcacgggtgccaatgtgatct

SEQ ID NO: 5 - DNA, 25 bp, Artificial Sequence
  LoopF primer
ggactgagatctttcattttaccgt

SEQ ID NO: 6 - DNA, 21 bp, Artificial Sequence
  LoopB primer
actgagggagccttgaataca

SEQ ID NO: 7 - DNA, 21 bp, Artificial Sequence
  F3 primer
caatggcttgtcttgtaggct

SEQ ID NO: 8 - DNA, 18 bp, Artificial Sequence
  B3 primer
agtcacctgctacacgct

SEQ ID NO: 9 - DNA, 66 bp, Artificial Sequence
  FIP primer
agtcacctgctacacgcttagaagcggtctggtcagaatagtgttccatgtggtcattcaatccag

SEQ ID NO: 10 - DNA, 42 bp, Artificial Sequence
  BIP primer
tgatccttcgtggacatcttcgtaggcaggtccttgatgtca

SEQ ID NO: 11 - DNA, 22 bp, Artificial Sequence
  LoopF primer
gtggcacgttgagaagaatgtt

SEQ ID NO: 12 - DNA, 21 bp, Artificial Sequence
  LoopB primer
ttgctggacaccatctaggac

SEQ ID NO: 13 - DNA, 28 bp, Artificial Sequence
  B3 primer
ggaatggtctgtgtttaatttatagttg

SEQ ID NO: 14 - DNA, 51 bp, Artificial Sequence
  BIP primer
cgtaatcggagctgtgatccttctcgtgatgtagcaacagtgatttcttta

SEQ ID NO: 15 - DNA, 21 bp, Artificial Sequence
  LoopB primer
tgtgacatcaaggacctgcct

SEQ ID NO: 16 - DNA, 48 bp, Artificial Sequence
  FIP HEX
tagaagcggtctggtcagaatagtgttccatgtggtcattcaatccag

SEQ ID NO: 17 - DNA, 25 bp, Artificial Sequence
  Pset 9 long Q
cactattctgaccagaccgcttcta

(19) United States
(12) Patent Application Publication
(10) Pub. No.: US 2024/0076752 A1
(43) Pub. Date: Mar. 7, 2024

(54) METHODS FOR DETECTION OF NUCLEIC ACIDS

(71) Applicants: March Therapeutics, Inc., Cambridge, MA (US); DxLab Inc., Somerville, MA (US)

(72) Inventors: Fahim Farzadfard, Boston, MA (US); Asmamaw Wassie, Boston, MA (US); Jeong Seuk Kang, Cambridge, MA (US); Ho-Jun Suk, Cambridge, MA (US); Kayla Vodehnal, Sunnyvale, CA (US); Justin Liu, Linwood, NJ (US); Jianghong Min, Cambridge, MA (US)

(21) Appl. No.: 18/260,447

(22) PCT Filed: Jan. 5, 2022
(86) PCT No.: PCT/US2022/011241

§ 371 Date: Jul. 5, 2023

Related U.S. Application Data
(60) Provisional application No. 63/199,515, filed on Jan. 5, 2021.

(51) Int. Cl. C12Q 1/70

(52) U.S. Cl. CPC C12Q 1/701

(57) ABSTRACT

The invention provides methods, kits, and related compositions for rapid, low-cost, point of care detection of an organism, such as a virus or bacteria, in a biological or environmental sample using isothermal nucleic acid amplification.

### RELATED APPLICATIONS

This application claims the benefit of priority under 35 U.S.C. § 119(e) to U.S. Provisional Application No. 63/199,515 filed Jan. 5, 2021, the entire contents of which is incorporated herein by reference in its entirety.

### FIELD OF THE INVENTION

The disclosure relates to methods and kits for rapid detection of pathogen associated nucleic acids directly in biological or environmental samples.

### INCORPORATION BY REFERENCE OF SEQUENCE LISTING

The contents of the sequence listing text file named “057930-501001WO_Sequence_Listing.txt”, which was created on Dec. 28, 2021 and is 4,096 bytes in size, is hereby incorporated by reference in its entirety.

### BACKGROUND OF THE INVENTION

In 2019 a new coronavirus named Severe Acute Respiratory Syndrome-CoV-2 s(SARS-CoV-2) was identified as the causative agent of a severe acute respiratory infection named coronavirus disease 2019 (COVID-19), which is causing a worldwide pandemic. The presence of the virus in subjects is determined by the detection of the RNA genome of the SARS-CoV-2 virus. Rapid point-of-care (POC) SARS-CoV-2 tests with good performance characteristics (e.g. sensitivity and specificity) and capable of operating in any low-resource setting, including at home, are needed to adequately combat the COVID-19 pandemic and reopen society.

Currently, a multitude of diagnostic tests for the detection and monitoring of the spread of SARS-CoV-2 have been developed. However, in the United States, most Center for Disease Control (CDC)-certified SARS-CoV-2 real-time reverse transcription PCR (RT-qPCR) diagnostic tests can only be effectively performed in a laboratory and have a turnaround time of approximately 4-6 hours, with results that can be delayed for more than 24 hours after sample collection due to shipping requirements. In order to achieve sensitive detection with the current technologies, the viral RNA needs to be extracted and purified from samples, making these methods unsuitable for truly POC applications. Furthermore, methods that rely on RT-qPCR require a thermocycler and relatively expensive reagents as well as a high level technical expertise, which may not be available in low-resource settings and POC, and therefore prohibit widespread use of these tests.

Therefore scalable, affordable, and easy-to-use POC tests remain an urgent need for the fight again COVID-19. The present invention addresses this need.

### SUMMARY OF THE INVENTION

The present invention provides methods and related compositions and kits for detecting an organism in a biological sample of a subject, preferably a human subject. In embodiments, the methods comprise subjecting the biological sample to chemical lysis by contacting the sample or an apparatus comprising the sample with a lysis buffer comprising a buffering agent, an RNase inhibitor, and a non-ionic surfactant to produce a lysed sample; subjecting the lysed sample to an isothermal nucleic acid amplification reaction performed in a reaction mixture comprising (i) a first set of 4-6 oligonucleotide primers directed to a first target nucleic acid which is a nucleic acid of the organism, (ii) a second set of 4-6 oligonucleotide primers directed to a second target nucleic acid which is a nucleic acid of the human subject, (iii) at least two different reagents suitable for independent detection of amplified target nucleic acids, (iv) a mixture of deoxyribonucleotide triphosphates (dNTPs), (v) a source of magnesium ions, (vi) an optional reverse transcriptase, and (vii) a DNA polymerase or a dual specificity reverse transcriptase/DNA polymerase, the method comprising detecting amplified target nucleic acids in the reaction mixture by detecting a signal from each of the at least two different reagents for detection of amplified target nucleic acids, wherein detection of at least two different signals indicates presence of the organism in the sample. In embodiments, the detection of at least two different signals occurs within a period of time of from 5-30 minutes.

In the context of the present invention, the term “lysed sample” refers to the lysed sample obtained by contacting the sample, or a collection apparatus containing the sample, such as an applicator or swab, with a buffer comprising reagents for chemical lysis.

In embodiments, the isothermal nucleic acid amplification is a loop-mediated isothermal nucleic acid amplification reaction (LAMP) or RT-LAMP reaction.

In embodiments, the at least two different reagents suitable for independent detection of amplified target nucleic acids comprises at least one set of detection primers adapted to contain a quencher-fluorophore duplex region, wherein the set of detection primers is directed to either the first or second target nucleic acid. In embodiments, the at least two different reagents suitable for independent detection of amplified target nucleic acids comprises two sets of detection primers, each adapted to contain a quencher-fluorophore duplex region, wherein each fluorophore emits a signal at a different wavelength and each set of detection primers is directed to a different target nucleic acid

In embodiments, the second target nucleic acid is a polynucleotide of a human gene that is constitutively expressed in human cells. In embodiments, human gene is selected from an actin gene, a ubiquitin gene, or other suitable housekeeping gene. In embodiments the polynucleotide is an RNase P or beta-actin DNA or RNA.

In embodiments, the one or more reagents for detection of the amplified target nucleic acid comprises a fluorescent DNA intercalating dye, optionally selected from the group consisting of Chai Green™, SYBR Green I and II, SYBR Safe, SYBR Gold, Eva Green, Ethidium Bromide, Oxazole yellow-based cyanine dyes (e.g. YOYO-1, DiYO-1, TOTO-1, DiTO-1, TOTO-3), Pico Green, SYTO 9, SYTO 13, SYTO 16, SYTO 60, SYTO 62, SYTO 64, SYTO 82, Boxto, Miami Green, Miami Yellow, and Miami Orange.

In embodiments, the reaction mixture further comprises one or more of an RNase inhibitor, a serum albumin (e.g, bovine serum albumin “BSA”), a reducing agent such as tris(2-carboxyethyl)phosphine (TCEP) and salts thereof, e.g., TCEP-HCl, and thermolabile uracil-DNA glycosylase (UDG).

In embodiments, the reaction mixture is lyophilized. In embodiments, the method further comprises a step of reconstituting the reaction mixture in a volume of aqueous solution. In embodiments, the aqueous solution utilized to reconstitute the lyophilized reaction mixture comprises the lysed sample, as described herein. In embodiments, the reaction mixture further comprises one or more of dextran, mannitol, sorbitol, maltodextrin, trehalose, lactose, and/or lactitol. In embodiments, the reaction mixture comprises one or more of trehalose, lactose, and/or lactitol.

In embodiments, the isothermal nucleic acid amplification reaction takes place at a constant temperature which is selected from a temperature in the range of 60-75° C., or from about 60-72° C., or preferably 63-69° C., or from about 64-69° C. For example, the nucleic acid amplification takes place at a temperature of 60° C., 61° C., 62° C., 63° C., 64° C., 65° C., 66° C., 67° C., 68° C., 69° C., 70° C., 71° C., or 72° C.

In embodiments, the method is performed within a period of time from 10 to 30 minutes.

In embodiments, the method further comprises one or more additional steps prior to performing the isothermal nucleic acid amplification reaction, the one or more additional steps comprising one or more of a clarification step comprising simultaneous ultrafiltration and concentration to produce a clarified sample, a chemical lysis step, and/or thermal treatment. In embodiments, the method comprises a chemical lysis step.

In embodiments, the clarification step comprises centrifugation at a speed of from about 10,000 to 100,000×g, or from 10,000 to 20,000×g, or from 14,000 to 15,000×g, for from 5 to 20 minutes.

In embodiments, the clarification step comprises ultrafiltration through a filter comprising a membrane having a molecular weight cut-off (MWCO) of from about 1 kDa to 300 kDa, preferably 3 kDa to 200 kDa, most preferably 100 kDa to 200 kDa.

In embodiments, the chemical lysis comprises contacting the sample with a lysis buffer, optionally wherein the lysis buffer comprises an RNase inhibitor, optionally polyvinylsulfonic acid (PVSA).

In embodiments, the lysis buffer comprises a lysis reagent selected from a non-ionic surfactant, guanidine hydrochloride, and guanidine thiocyanate. In embodiments, the lysis reagent is a non-ionic surfactant. In embodiments, the non-ionic surfactant is selected from a polyethylene glycol p-(1,1,3,3-tetramethylbutyl)-phenyl ether (Triton™ X-100) or similar Triton™ detergent, or a polysorbate-type nonionic surfactant such as polyoxyethylene (20) sorbitan monolaurate (Tween™ 20) or similar Tween™ detergent such as Tween 80, or a maltoside such as n-dodecyl-β-D-maltoside.

In embodiments, the thermal treatment comprises heating the sample to about 95° C. for a period of time from 30 seconds to 10 minutes, preferably 30 seconds to 5 minutes, most preferably 1 minute to 3 minutes.

In embodiments, the target nucleic acid comprises one or more RNA or DNA molecules of an organism selected from a virus, a bacterium, a fungus or a parasite.

In embodiments, the target nucleic acid is of a virus.

In embodiments, the virus is selected from severe acute respiratory syndrome coronavirus (SARS-CoV), Middle East respiratory syndrome coronavirus (MERS-CoV), and severe acute respiratory syndrome coronavirus 2 (SARS-CoV-2).

In embodiments, the virus is SARS-Cov-2.

In embodiments, the first set of oligonucleotide primers is selected from the group consisting of Set 1, Set 2, Set 3, and Set 4, identified by SEQ ID NOs 1-6 (Set 1), SEQ ID NOs 7-12 (Set 2), SEQ ID NOs 7, 9, 11, 13-15 (Set 3), and SEQ ID NOs 7-12, 16, 17 (Set 4). In embodiments of the method where the virus is SARS-Cov-2, the set of 4-6 oligonucleotide primers is selected from the group consisting of Set 1, Set 2, Set 3, and Set 4, identified by the sequence identifiers set forth in Table 1, absent the LoopF and LoopB primers, i.e., SEQ ID NOs 1-4 of Set 1, SEQ ID NOs 7-10 of Set 2, SEQ ID NOs 7, 13, 9, and 14, of Set 3, or SEQ ID NOs 7-10 and SEQ ID NOs 16 and 17 of Set 4. In embodiments, the set of 4-6 oligonucleotide primers hybridizes to gene N or gene M of SARS-CoV-2 viral RNA.

In embodiments, the biological sample is an upper respiratory tract sample, optionally wherein the upper respiratory tract sample is obtained from a nasopharyngeal (NP) swab, an oropharyngeal (OP) swab, a nasal swab, a nasal aspirate, or a nasal wash, further optionally wherein the nasal swab is an anterior nasal swab or a mid-turbinate nasal swab.

Also provided is a method for detecting a SARS-CoV-2 virus in an upper respiratory tract sample obtained from a human subject, the method comprising subjecting the sample to a reverse transcription and loop-mediated isothermal nucleic acid amplification reaction (RT-LAMP) to amplify a first target nucleic acid of the SARS-CoV-2 virus utilizing a first set of oligonucleotide primers selected from the group consisting of Set 1, Set 2, Set 3, and Set 4, identified by SEQ ID NOs 1-6 (Set 1), SEQ ID NOs 7-12 (Set 2), SEQ ID NOs 7, 9, 11, 13-15 (Set 3), and SEQ ID NOs 7-12, 16, 17 (Set 4), and detecting the amplified first target nucleic acid, wherein detection of the amplified first target nucleic acid indicates presence of the SARS-CoV-2 virus in the biological sample.

In embodiments, the RT-LAMP reaction is performed in a reaction mixture comprising the first set of oligonucleotide primers and a second set of oligonucleotide primers directed to a second target nucleic acid which is a nucleic acid of the human subject, optionally wherein the second target nucleic acid is a beta-actin or RNase P RNA or DNA.

In embodiments, the reaction mixture further comprises a set of detection primers adapted to contain a quencher-fluorophore duplex region, wherein the set of detection primers is directed to either the first or second target nucleic acid.

In embodiments, the reaction mixture further comprises a fluorescent DNA intercalating dye that emits a fluorescent signal at a different wavelength than the fluorophore in the detection primer.

In embodiments, the reaction mixture further comprises a mixture of deoxyribonucleotide triphosphates (dNTPs), a source of magnesium ions, a reverse transcriptase, a DNA polymerase or a dual specificity reverse transcriptase/DNA polymerase, and one or more of an RNase inhibitor, a serum albumin, a reducing agent such as tris(2-carboxyethyl)phosphine (TCEP) and salts thereof, e.g., TCEP-HCl, and thermolabile uracil-DNA glycosylase (UDG).

In embodiments, the reaction mixture is lyophilized and the method further comprises a step of reconstituting the reaction mixture in a volume of an aqueous solution. In embodiments, the aqueous solution is comprises the lysed sample, as described herein, optionally wherein the lyophilized reaction mixture comprises one or more of dextran, mannitol, sorbitol, maltodextrin, trehalose, lactose, and/or lactitol. In embodiments, the lyophilized reaction mixture comprises one or more of trehalose, lactose, and/or lactitol.

Also provided is a kit for detection of a SARS-CoV-2 virus, the kit comprising (i) a first container comprising a first set of reagents for sample elution and lysis, and (ii) a second container comprising a second set of reagents for performing a reverse transcription and loop-mediated isothermal nucleic acid amplification reaction (RT-LAMP). In embodiments, the second set of reagents comprises a first set of oligonucleotide primers directed to a first target nucleic acid of the SARS-CoV-2 virus selected from the group consisting of Set 1, Set 2, Set 3, and Set 4, identified by SEQ ID NOs 1-6 (Set 1), SEQ ID NOs 7-12 (Set 2), SEQ ID NOs 7, 9, 11, 13-15(Set 3), and SEQ ID NOs 7-12, 16, 17 (Set 4); and a second set of oligonucleotide primers directed to a second target nucleic acid of the human subject. In embodiments, the second target nucleic acid is a polynucleotide of a human gene that is constitutively expressed in human cells. In embodiments, the human gene is selected from an actin gene, a ubiquitin gene, or other suitable housekeeping gene. In embodiments the polynucleotide is an RNase P or beta-actin DNA or RNA molecule. In embodiments, the first set of reagents comprises an RNase inhibitor and a chemical lysis reagent. In embodiments, the second set of reagents further comprises deoxyribonucleotide triphosphates (dNTPs) including dUTP, a source of magnesium ions (e.g., MgCl₂ or MgSO₄), a reverse transcriptase, and a DNA polymerase. In embodiments, the second set of reagents further comprises a fluorescent DNA intercalating agent and a set of detection primers adapted to contain a quencher-fluorophore duplex region, wherein the set of detection primers is directed to either the first or second target nucleic acid. In embodiments, the second set of reagents comprises two sets of detection primers, each adapted to contain a quencher-fluorophore duplex region, wherein each fluorophore emits a signal at a different wavelength and each set of detection primers is directed to a different target nucleic acid. In embodiments, the second set of reagents further comprises one or more of an RNase inhibitor, a serum albumin, a reducing agent such as tris(2-carboxyethyl)phosphine and salts thereof, e.g., TCEP-HCl, and thermolabile uracil-DNA glycosylase (UDG). In embodiments, the second set of reagents is lyophilized. In embodiments, the second set of reagents further comprises one or more of dextran, mannitol, sorbitol, maltodextrin, trehalose, lactose, and/or lactitol. In embodiments, the second set of reagents further comprises one or more of trehalose, lactose, and/or lactitol.

### BRIEF DESCRIPTION OF THE FIGURES

FIGS. 1A-C: Line plot showing RT-LAMP amplification profiles for (A) test primer sets i-viii, with primer set 1 indicated by arrows; (B) primer sets ix-xvi, with primer set 2 indicated by arrows; and (C) primer set 3. y-axis depicts amplified product measured as relative fluorescence units; x-axis depicts 1-minute cycle number from 0-120. Cq values for primer sets 1, 2, and 3 were 42, 37, and 35, respectively. The Cq value is the time needed to reach half maximum height.

FIGS. 2A-B: Cross-reactivity of RT-LAMP SARS-CoV-2 primers towards other respiratory viruses for primer sets 1 (A) and 3 (B). Each reaction was performed in 4 replicates; the four lines in each panel showing early amplification (between cycles 15-25 in panel A and between cycles 25-35 in panel B) represent amplification profiles for SARS-CoV-2; y-axis depicts amplified product measured as relative fluorescence units; x-axis depicts 1-minute cycle number from 0-90. Each of primers sets 1 and 3 demonstrated specific amplification towards SARS-CoV-2 enabling the discrimination of SARS-CoV-2 samples from other respiratory viruses.

FIG. 3: Limit of detection (LOD) of the inactivated SARS-CoV-2 viral particles by RT-LAMP with a lysis step. Normal saline containing saliva and RiboGuard™ was spiked with either 50 (left whisker plot, light shade) or 100 (right whisker plot, darker shade) virions/microliter of inactivated SARS-CoV-2 virions, the samples were then lysed in QuickExtract™ Buffer (3 min, 95 C). Lysed samples were used as a template in RT-LAMP reactions.

FIG. 4: Centrifugal filtration enables increases sensitivity of detection of SARS-CoV-2 with RT-LAMP. Plot shows the performance of RT-LAMP with filtration (right boxes) or without (left boxes) with either 10 (left whisker plot, light shade) or 20 (right whisker plot, dark shade) virions/microliter of inactivated SARS-CoV-2 virions spiked into saline samples (n=3, s.t.d). Normal Saline containing saliva and ribonuclease inhibitor (RiboGuard™) was spiked with different concentrations of inactivated SARS-Cov-2 virions, the samples were then lysed by heat-treatment in QuickExtract Buffer (3 min, 95 C). Lysed samples were used as a template in RT-LAMP reactions

FIG. 5: Limit of detection (LOD) of the inactivated SARS-CoV-2 viral particles by RT-LAMP showing Ct time (mill) for assays in which the centrifugal filtration step was performed (left bars in each pair) compared to assay without that step (right bars in each pair). Normal saline containing saliva and ribonuclease inhibitor (RiboGuard™) was spiked with inactivated SARS-CoV-2 virions in concentrations corresponding to 1 copy/μl, 5 copies/μl, and 20 copies/μl. No-template control (NTC) was used as the negative control. Error bars indicate standard deviation for four replicates. These results show that LoD=1 copy/μl can be achieved by incorporation of the filtration step into the protocol.

FIG. 6: Representative components of test kit comprising a buffer tube (A), a sterile swab (B), and a test pouch (C) containing a transfer pipette (C1) and a tube assembly (C2).

FIG. 7A-D: Amplification curves of negative and positive samples with or without nasal swab sample. A, without a nasal swab sample, fast FAM channel amplification (T_{d}<26 min.) as seen in the 1 viral copy/μL tests, represents the presence of SARS-CoV-2 virus. Late FAM channel amplification (T_{d}>26 min.) signifies false amplification and is disregarded. B, without a nasal swab sample, fast HEX channel amplification (T_{d}<26 min.), as seen in the 1 viral copy/μL tests, represents the presence of SARS-CoV-2 virus. Late HEX channel amplification (T_{d}>26 min.) signifies false amplification and is disregarded. Absence of both FAM and HEX channel amplification within 26 minutes indicates absence of virus in the sample. C, with a nasal swab sample, fast FAM channel amplification (T_{d}<26 min.) represents the general presence of beta actin and/or SARS-CoV-2 virus. D, with a nasal swab sample, fast HEX channel amplification (T_{d}<26 min.), as seen in the 1 viral copy/μL tests, represents the presence of SARS-CoV-2 virus. Fast FAM channel amplification (T_{d}<26 min.) with late or no HEX channel amplification signifies a nasal swab sample with no detectable SARS-CoV-2 virus. T_{d}: Time at which a decision to call a test “positive” or “negative” is made by our test algorithm; FAM: Fluorophore with a peak absorption wavelength of 495 nm and a peak emission wavelength of 520 nm; HEX: Fluorophore with a peak absorption wavelength of 535 nm and a peak emission wavelength of 556 nm.

FIG. 8: Limit of Detection (LOD) study. Range finding experiments were performed for 1500, 3000, and 6000 viral copies/swab. There was 1 sample with 1500 copies/swab and 1 sample with 3000 copies/swab that had a HEX channel T_{d}>26 min., which is called “negative” by our test algorithm. 3000 copies/swab was determined to be the LOD after 22 out of 23 replicates were correctly called “positive” by our test algorithm (>95%, threshold set by the U.S. Food and Drug Administration). T_{d}: Time at which a decision to call a test “positive” or “negative” is made by our test algorithm; FAM and HEX are as defined in the legend to FIG. 7 above.

### DETAILED DESCRIPTION OF THE INVENTION

The disclosure provides methods and related compositions suitable for the rapid detection of an organism, such as a pathogen, e.g., a virus, bacterium, or other pathogen, directly in a biological or environmental sample, using an isothermal nucleic acid amplification reaction and at least one set of oligonucleotide primers directed to a nucleic acid of the organism, for example a particular RNA or DNA molecule of the organism. In addition, the methods provide for the simultaneous detection of two or more amplified nucleic acid targets in a single reaction mixture using a combination of detectable DNA intercalating agents, such as a fluorogenic DNA intercalating dye, and one or more sets of detection primers which produce a detectable signal, such as fluorescence, upon target sequence amplification. In embodiments of the methods described here, the method provides for the detection of a nucleic acid of the organism and the simultaneous detection of a human nucleic acid, such as actin RNA or similar ubiquitously expressed human RNA.

The methods described here comprise oligonucleotide directed amplification of one or more target nucleic acids using an isothermal nucleic acid amplification reaction such as a loop-mediated isothermal nucleic acid amplification reaction (LAMP), High-Performance LAMP (HP-LAMP), Reverse Transcription Loop-mediated Isothermal Amplification (RT-LAMP), and other similar reactions as described infra, and appropriate oligonucleotide primers. In this context a “primer” refers to a short, usually about 15-60 nucleotides in length (or from about 15-25 nucleotides in length), single-stranded RNA or DNA sequence whose sequence or certain portions of the sequence are complementary to and therefore hybridizes with a target nucleic acid via Watson-Crick base pairing. For example, it is understood in the context of LAMP reactions that the forward inner primers (FIP) and backward inner primers (BIP) primers contain bridging regions that are not complementary to the target nucleic acid. In the context of the methods described here, a primer “directed to” a target nucleic acid is one that is complementary to a sequence of the target nucleic acid and a “primer set” refers to a pair of primers targeted to direct DNA elongation toward each other at opposite ends of the target nucleic acid sequence being amplified.

A “detection primer” refers to a primer that produces a detectable signal useful to detect an amplification product of a target nucleic acid. In a preferred embodiment, the detection primers consist of LAMP or RT-LAMP primers adapted to contain a quencher-fluorophore duplex region that emits a fluorescent signal upon strand separation, thereby allowing for detection of the amplification product of the nucleic acid targeted by the primers. These modified primers are referred to as “DARQ” primers (“Detection of Amplification by Releasing of Quenching”) and are described in Tanner N A et al., Biotechniques (2012) 53:81-89. Briefly, a DARQ primer set consists of a pair of oligonucleotides primers, one long and one short. The longer primer is between about 30-60 nucleotides long and the shorter primer is about 15-30 nucleotides long. The shorter primer is completely complementary to the longer primer, and the portion of the longer primer that is not complementary to the shorter primer is complementary to a portion of a target nucleic acid, e.g., DNA or RNA. Each DARQ primer is modified with either a fluorophore or a quencher, on opposite ends of the oligonucleotide sequence (for example, a fluorophore at the 5′ end for the longer primer and a quencher at the 3′ end for the shorter primer). This configuration keeps the fluorophore and quencher in close proximity to one another when the primers are base-paired together as a double-helix. In this configuration, the quencher prevents the fluorophore from emitting a detectable signal. As LAMP or RT-LAMP amplification occurs, the longer DARQ primer is incorporated into the amplification product and the shorter DARQ primer is displaced from the longer primer, separating the fluorophore from the quencher, and permitting the fluorophores to emit a detectable signal. In embodiments, DARQ primers are used in the present methods to specifically identify successful amplification of a primer set they are paired with, for example primer set 4 described in Table 1.

In embodiments of the methods described here, more than one set of detection primers may be used, for example to detect the amplification of two different target nucleic acids. In accordance with such embodiments, each different set of detection primers emits a signal, such as colorimetric or fluorescence signal, at a wavelength different from that of any other set of detection primers in the same assay in order to provide for the independent detection of each amplified target nucleic acid. In some embodiments, where one or more sets of fluorescent detection primers is present, each emits a signal at a wavelength that is also different from the wavelength emitted by any fluorogenic DNA intercalating dye included in the reaction, such that simultaneous detection of each independent signal is achieved. The simultaneous detection of multiple amplified target nucleic acids, advantageously provides for the option to include internal controls for assay fidelity, e.g., by detection of a constitutively expressed human gene. For example, a second primer set targeted to a human RNA may be included to ensure that an adequate amount of a human cellular material was collected in the biological sample being tested. In embodiments, the second primer set targets the RNA of a human ‘housekeeping’ gene or other human gene that is constitutively expressed at a relatively high abundance in human cells. This ensures that every biological sample collected contains sufficient genetic material for pathogen testing and diagnostics, and serves to reduce false negative results generated, for example, from under collection of a biological sample.

Advantageously, the methods described herein can be practiced without the need for a standard nucleic acid purification step, such as solid-phase extraction with a silica matrix, or a step of phenol-chloroform extraction or cold alcohol precipitation. Instead, the isothermal nucleic acid amplification reaction can be performed following chemical lysis of cells obtained from a biological sample, for example by placing the tip of a collection apparatus, such as a swab, in a buffer for sample elution and lysis to produce a lysed sample, as described herein. In embodiments, the elution and lysis buffer comprises a surfactant, preferably a non-ionic surfactant, such as Triton™ X-100 or similar Triton™ detergent, or a Tween™ detergent such as Tween 20 or Tween 80, or a maltoside such as n-dodecyl-β-D-maltoside; a buffering agent, such as Tris, CAPS, HEPES, MOPS, PIPES, TAPS, TE, TES, or Tricine; an RNase inhibitor, such as polyvinylsulfonic acid (PVSA). In some embodiments, the methods comprise a clarification step comprising simultaneous ultrafiltration and concentration to produce a clarified sample, prior to isothermal nucleic acid amplification.

As used herein, the terms “detecting” and “detection” involve quantitative and/or qualitative determinations. This term is intended to exclude purely mental steps and instead refer to the use of one or more laboratory assays for determining the presence of an organism (e.g., SARS-CoV-2) in a sample. Such methods may include computer assisted steps for determining the amount of an analyte, such as the amount of a nucleic acid, e.g., RNA or DNA, or the amount of a protein, peptide, or polypeptide, in the sample, including for example the amount of an amplified target nucleic acid in the sample.

In some embodiments, the detection of the target nucleic acid in the sample has a specified limit of detection (LOD). In the context of the methods described here, LOD is the lowest concentration of target nucleic acid that can be detected in greater than or equal to 95% of repeat measurements. In accordance with generally accepted standards, LOD may be described as units of copies of genomic RNA or DNA per microliter of transport media, or in specific embodiments, as copies of viral genomic RNA per microliter of transport media (copies/μL). For clinical applications, the LOD is preferably in the range of 0.1-10 copies/μL, or from about 0.1-1 copies/μL (or about 100-10,000 or 100-1,000 copies/milliliter). In some embodiments described here, LOD may be defined as the number of copies of genomic RNA, or viral particles, per unit of volume, or per reaction. In some aspects, the LOD may be described in terms of a “genome equivalent” which is defined as the amount of DNA necessary to be present in a purified sample to guarantee that all genes will be present. In other embodiments, the LOD may be defined in terms of Nucleic Acid-based Amplification Test (NAAT) Detectable Units (NDUs) per milliliter of sample. In some embodiments, the NDU is from about 10 to 2000, 50 to 500, or 100 to 300 NDUs/ml of sample. In other embodiments, the NDU/mL is from about 600-1800, or greater than 1800 NDU/mL. For viral genomes of small size, such as that of SARS-CoV-2, LOD and NDU are essentially equivalent.

In certain embodiments, the methods described here have a sensitivity of from about 90% to about 100%, preferably from about 95% to about 100%, most preferably at least about 97%. As used herein, “sensitivity” is defined as the number of positive control samples (i.e., samples known to contain a specific target nucleic acid) identified by a nucleic acid detection method as positive (e.g., if 29 out of 30 positive control samples is identified by a nucleic acid detection method as positive, then said detection method has a sensitivity of 97%).

In embodiments, the methods described herein have a specificity of from about 90% to about 100%, preferably from about 95% to about 100%, most preferably at least about 99%. As used herein, the term “specificity” is defined as the number of negative control samples (i.e, samples known to not contain a specific target nucleic acid) identified by a nucleic acid detection method as negative (e.g., if 29 out of 30 negative control samples is identified by a nucleic acid detection method as negative, then said detection method has a specificity of 97%). Therefore, specificity is a measure of the absence of non-specific amplification in the sample.

A sample used in accordance with the methods described here can be any sample containing genetic material, e.g., any sample containing DNA or RNA or both.

In embodiments, the sample is an environmental sample. In certain aspects, the environmental sample includes, but is not limited to, particles collected from a solid surface, a liquid, and a volume of air.

In other embodiments, the sample is a biological sample. The term “biological sample” as used herein may refer to a sample, including a biopsy sample, of a tissue, or other biological sample such as an exudate, gastric lavage, saliva, serum, plasma, mucus, blood, or urine sample; or a swab such as an oral swab, a buccal swab, a nasal swab, a pharyngeal swab, an oropharyngeal swab, a mid-turbinate swab, or a nasopharyngeal swab taken from a subject. In some embodiments, the sample is a tissue sample, for example a lung tissue sample, a gastric or gastric mucosal tissue sample, or a blood sample, including a serum or plasma sample. In certain aspects, the biological sample includes, but is not limited to, saliva, mucus, blood, or serum, obtained from a subject. In specific aspects, the biological sample is an upper respiratory tract sample. In embodiments, the upper respiratory tract sample is obtained from a nasopharyngeal (NP) swab, an oropharyngeal (OP) swab, a nasal swab, a nasal aspirate, or a nasal wash. In embodiments, the nasal swab is an anterior nasal swab or a mid-turbinate nasal swab.

The term “isothermal nucleic acid amplification reaction” refers to any nucleic acid amplification technique that takes place at a constant temperature. Thus, in contrast to other nucleic acid amplification technologies (e.g., polymerase chain reaction or PCR), in which the reaction is carried out with a series of alternating temperature steps or cycles, isothermal nucleic acid amplification is carried out at a constant temperature, and does not require a thermal cycler. In embodiments, the isothermal nucleic acid amplification reaction takes place at a constant temperature that may be selected from a temperature in a range of from 40-100° C., from 50-72° C., from 55-65° C., or from 55-70° C., or from 60-72° C. In specific embodiments, the isothermal nucleic acid amplification reaction takes place at a constant temperature of 60° C., 61° C., 62° C., 63° C., 64° C., 65° C., 66° C., 67° C., 68° C., 69° C., 70° C., 71° C., or 72° C. In an embodiment, the isothermal nucleic acid amplification reaction takes place at a constant temperature of 65 or 67° C.

In embodiments, the isothermal nucleic acid amplification reaction may be a loop-mediated isothermal nucleic acid amplification reaction (LAMP), High-Performance LAMP (HP-LAMP), Reverse Transcription Loop-mediated Isothermal Amplification (RT-LAMP), mismatch-tolerant RT-LAMP, One-pot RT-LAMP, integrated RT-LAMP and CRISPR-Cas12 method, SHERLOCK (Specific High Sensitivity Enzymatic Reporter Unlocking), STOP (SHERLOCK Testing in One Pot), isothermal LAMP based method for COVID-19 (iLACO), and Penn-RAMP. In embodiments, the isothermal nucleic acid amplification reaction may be a whole genome amplification (WGA), strand displacement amplification (SDA), Nicking Enzyme Amplification Reaction (NEAR), helicase dependent amplification (HDA), recombinase polymerase amplification (RPA), Nucleic Acid Sequenced Based Amplification (NASBA), or a Transcription Mediated Amplification (TMA).

In embodiments, the isothermal nucleic acid amplification is a loop-mediated isothermal nucleic acid amplification reaction (LAMP). In specific aspects, the target nucleic acid is an RNA and the LAMP further comprises reverse transcription (RT-LAMP). In an embodiment, the RT-LAMP or LAMP reaction takes place at a constant temperature of 65 or 67° C.

In certain embodiments, the RT-LAMP or LAMP takes place in a reaction mixture comprising at least one set of 4-6 oligonucleotide primers. In embodiments, the set of oligonucleotide primers is selected from a set of primers described in Table 1. In embodiments, the set of oligonucleotide primers is selected from a set of primers described in Table 1 except the set does not comprise the LoopF and/or LoopB primers, thereby providing a set of 4 primers. In embodiments, the at least one set of 4-6 oligonucleotide primers hybridizes to a target nucleic acid of the organism. In embodiments, the reaction mixture further comprises at least one additional set of 4-6 oligonucleotide primers that hybridize to a target human nucleic acid, such as beta-actin or similar human gene that is constitutively expressed in human cells. RT-LAMP and LAMP primer sets directed against suitable human nucleic acid targets are known in the art, for example the beta actin primer set described by Zhang and Tanner (2020). In embodiments of the methods described here, at least one of the set of 4-6 oligonucleotide RT-LAMP or LAMP primers is paired with a DARQ primer set targeting the same nucleic acid. In some embodiments, each of the RT-LAMP or LAMP primer sets is paired with its own DARQ primer set, wherein each unique DARQ primer set is adapted to emit a fluorescent signal at a different wavelength in order to allow for the simultaneous detection of two or more amplification products in a single reaction mixture.

In embodiments, the at least one set of 4-6 oligonucleotide primers hybridizes to a specific gene of SARS-CoV-2 viral RNA including, but not limited to, gene N (encoding Sars-Cov-2 nucleoplasmid), gene M (encoding Sars-Cov-2 membrane protein), gene E (encoding Sars-Cov-2 envelope protein), gene S (encoding Sars-Cov-2 spike protein), ORF1ab (encoding orf1 abpolyproteins), Orf3a (encoding accessory protein), Orf6a (encoding accessory protein), Orf7a (encoding accessory protein), Orf7b (encoding accessory protein), Orf8 (encoding accessory protein), and Otf10 (encoding accessory protein). In certain embodiments, the at least one set of 4-6 oligonucleotide primers hybridizes to gene N or gene M of SARS-CoV-2 viral RNA. In certain embodiments, the at least one set of 4-6 oligonucleotide primers is selected from the group consisting of Set 1, Set 2, Set 3 and Set 4, identified by SEQ ID NOs 1-6 (Set 1), SEQ ID NOs 7-12 (Set 2), SEQ ID NOs 7, 9, 11, 13-15 (Set 3), and SEQ ID NOs 7-12, 16, 17 (Set 4). The sequences of the oligonucleotide primers from sets 1, 2, 3 and 4 are shown in Table 1 below:

TABLE 1
Oligonucleotide Primer Sequences for Primer Sets 1-4.

| | | | |
|---|---|---|---|
| SEQ | | | |
| ID | | | |
| NO: | Role | Sequence | Target Gene |

Primer Set 1

| | | | |
|---|---|---|---|
| 1 | F3 primer | TGGCTACTACCGAAGAGCT | Gene N (Sars-Cov-2 |
| | | | nucleoplasmid) |
| 2 | B3 primer | TGCAGCATTGTTAGCAGGAT | Gene N |
| 3 | FIP primer | TCTGGCCCAGTTCCTAGGTAGTCCAGACGAA | Gene N |
| | | TTCGTGGTGG | |
| 4 | BIP primer | AGACGGCATCATATGGGTTGCACGGGTGCCA | Gene N |
| | | ATGTGATCT | |
| 5 | LoopF | GGACTGAGATCTTTCATTTTACCGT | Gene N |
| | primer | | |
| 6 | LoopB | ACTGAGGGAGCCTTGAATACA | Gene N |
| | primer | | |

Primer Set 2

| | | | |
|---|---|---|---|
| 7 | F3 primer | CAATGGCTTGTCTTGTAGGCT | Gene M (Sars-Cov-2 |
| | | | membrane protein) |
| 8 | B3 primer | AGTCACCTGCTACACGCT | Gene M |
| 9 | FIP primer | TAGAAGCGGTCTGGTCAGAATAGTGTTCCAT | Gene M |
| | | GTGGTCATTCAATCCAG | |
| 10 | BIP primer | TGATCCTTCGTGGACATCTTCGTAGGCAGGT | Gene M |
| | | CCTTGATGTCA | |
| 11 | LoopF | GTGGCACGTTGAGAAGAATGTT | Gene M |
| | primer | | |
| 12 | LoopB | TTGCTGGACACCATCTAGGAC | Gene M |
| | primer | | |

Primer Set 3

| | | | |
|---|---|---|---|
| 7 | F3 primer | CAATGGCTTGTCTTGTAGGCT | Gene M |
| 13 | B3 primer | GGAATGGTCTGTGTTTAATTTATAGTTG | Gene M |
| 9 | FIP primer | TAGAAGCGGTCTGGTCAGAATAGTGTTCCAT | Gene M |
| | | GTGGTCATTCAATCCAG | |
| 14 | BIP primer | CGTAATCGGAGCTGTGATCCTTCTCGTGATG | Gene M |
| | | TAGCAACAGTGATTTCTTTA | |
| 11 | LoopF | GTGGCACGTTGAGAAGAATGTT | Gene M |
| | primer | | |
| 15 | LoopB | TGTGACATCAAGGACCTGCCT | Gene M |
| | primer | | |

Primer Set 4

| | | | |
|---|---|---|---|
| 7 | F3 primer | CAATGGCTTGTCTTGTAGGCT | Gene M |
| 8 | B3 primer | AGTCACCTGCTACACGCT | Gene M |
| 9 | FIP primer | TAGAAGCGGTCTGGTCAGAATAGTGTTCCAT | Gene M |
| | | GTGGTCATTCAATCCAG | |
| 10 | BIP primer | TGATCCTTCGTGGACATCTTCGTAGGCAGGT | Gene M |
| | | CCTTGATGTCA | |
| 11 | LoopF | GTGGCACGTTGAGAAGAATGTT | Gene M |
| | primer | | |
| 12 | LoopB | TTGCTGGACACCATCTAGGAC | Gene M |
| | primer | | |
| 16 | FIP HEX | /5HEX/TAGAAGCGGTCTGGTCAGAATAGTGT | Gene M |
| | | TCCATGTGGTCATTCAATCCAG | |
| 17 | Pset 9 long | CACTATTCTGACCAGACCGCTTCTA/3bHQ_1/ | |
| | Q** | | |

*/5HEX/ indidates a 5′ Hexachlorofluorescein modification
**/3bHQ_1/ indicates a 3′ Black Hole Quencher®-1

In embodiments, the RT-LAMP or LAMP takes place in a reaction mixture comprising a set of 4-6 oligonucleotide primers directed to a target nucleic acid of the organism and a second set of 4-6 oligonucleotide primers directed to a human nucleic acid, for example a human RNA that is expressed consistently in all human cells, such as a human actin RNA, e.g., beta actin.

In certain embodiments, the RT-LAMP or LAMP reaction takes place in a reaction mixture in a closed container comprising one or more reagents for detection of the one or more amplified target nucleic acids. In embodiments, the one or more reagents for detection of the one or more amplified target nucleic acids comprises one or more fluorescent DNA indicators. The term “indicator” refers to a molecule that produces a detectable signal including, but not limited to, fluorescence, visible color, chemiluminescence, and radioactivity.

In some aspects, the one or more fluorescence DNA indicators include, but are not limited to, fluorescein isothiocyanate (FITC), Rhodamine X (ROX), 6-carboxyfluorescein (FAM), SYTO 9, SYTO 82, SYTO 16, SYTO 13, SYTO 64, Boxto, Miami Green, Miami Yellow, Miami Orange, YOPRO 1, SYTO 62, TOPRO 3, SYTO 60, Chai Green™, EvaGreen, POPO 3, NG-DCS 1, SYBR Green I, SYBR Green II, BOBO 3, TOTO 3, Pico 488, TOTO 1, and SYTO 24.

In embodiments, the one or more fluorescence DNA indicators is selected from a fluorescent DNA intercalating dye such as a SYBR green or Chai Green™, or similar dye such as SYBR Green I and II, SYBR Safe, SYBR Gold, Eva Green, Ethidium Bromide, Oxazole yellow-based cyanine dyes (e.g. YOYO-1, DiYO-1, TOTO-1, DiTO-1, TOTO-3), Pico Green, SYTO 9, SYTO 13, SYTO 16, SYTO 60, SYTO 62, SYTO 64, SYTO 82, Boxto, Miami Green, Miami Yellow, or Miami Orange.

In certain aspects, the detection of the amplified target nucleic acid comprises an increased fluorescent signal in the sample over time.

In embodiments, the one or more reagents for detection of the one or more amplified target nucleic acids comprises a non-fluorescent DNA indicator, such as a colorimetric indicator. In certain aspects, the colorimetric indicator may include one or more of malachite green, calcein and hydroxynaphthol blue.

In embodiments, the oligonucleotide primers of each primer set may be labeled with a different colorimetric or fluorescent indicator such that the amplification products of two or more different target nucleic acids may be detected simultaneously in the reaction mixture.

In embodiments, the reaction mixture may further comprise a pH-sensitive colorimetric indicator. The term “pH-sensitive indicator” refers to a molecule that produces a visible change in color (e.g., from red to yellow) in response to lowered pH of the reaction mixture as an isothermal nucleic acid amplification proceeds. In certain aspects, the one or more pH-sensitive colorimetric indicators include, but are not limited to, phenol red, cresol red, neutral red, bromocresol purple, and m-cresol purple.

The RT-LAMP or LAMP reaction mixture further comprises one or more enzymes selected from a reverse transcriptase, a DNA polymerase, and a dual specificity reverse transcriptase/DNA polymerase enzyme. Typically, a reverse transcriptase is active at about 37° C. to about 42° C. In some embodiments, the reverse transcriptase is a thermostable reverse transcriptase. Thermostable reverse transcriptases generally refer to the reverse transcriptases that retain part or all of their catalytic activities under elevated temperatures. Typically, a thermostable reverse transcriptase is active at or above 50° C. In some embodiments, the reverse transcriptase is a wild-type enzyme. In some embodiments, the reverse transcriptase comprises one or more single point mutations. In other embodiments, the reverse transcriptase is truncated. In some embodiments, the reverse transcriptase is an RNase H⁻ mutant.

Exemplary reverse transcriptase includes, without limitations, M-MLV reverse transcriptase, AMV reverse transcriptase, FeLV reverse transcriptase, ExcellScript Thermostable M-MuLV Reverse Transcriptase, RapiDxFire™ Thermostable Reverse Transcriptase, RocketScript™ Thermostable Reverse Transcriptase, Superscript II Reverse Transcriptase, Superscript III Reverse Transcriptase, Superscript IV Reverse Transcriptase, Protoscript® II Reverse Transcriptase, Maxima H minus Reverse Transcriptase, WarmStart® RTx Reverse Transcriptase. Different reverse transcriptases have different characteristics, and some are better suited to specific applications than others. The downstream application, the length of the target RNA, presence of complex RNA secondary structure and an enzyme's level of RNase H activity are all considerations when choosing the right reverse transcriptase.

In some embodiments, the DNA polymerase is a thermostable DNA polymerase. Thermostable DNA polymerase are commonly used in the art, and are able to catalyze the extension reaction on the DNA template. In some embodiments, the thermostable DNA polymerase comprises a Taq polymerase, a Tth Polymerase, and a Z05 polymerase. In some embodiments, the thermostable DNA polymerase is a wild-type enzyme. In other embodiments, the thermostable DNA polymerase comprises one or more single point mutations. In some embodiments, the thermostable DNA polymerase does not have the 5′-3′ exonuclease activity. In other embodiments, the thermostable DNA polymerase has the 5′-3′ exonuclease activity and is referred to as exo+ Taq DNA polymerase.

In some embodiments, the DNA polymerase is a strand displacing polymerase. A strand displacing polymerase catalyzes the extension reaction on the DNA template without the need for thermocycling, or adjusting the temperature of the reaction between 50° C. and 70° C. in a cyclic manner. Instead, strand displacing polymerases are able to remove the complementary DNA strand and replace it with a newly synthesized strand as it moves along the template DNA strand at a constant temperature. In embodiments, the DNA polymerase is both a strand displacing polymerase and a thermostable polymerase. Suitable examples include the Bst polymerase and Phi-29 polymerase.

In some embodiments, the RT-LAMP or LAMP reaction mixture further comprises deoxyribonucleotide triphosphates (dNTPs), which may be a mixture of dATP, dCTP, dGTP, dTTP, and dUTP. In additional embodiments, the RT-LAMP or LAMP reaction mixture further comprises a source of magnesium ions. In embodiments, the source of magnesium ions is one or both of MgCl₂ and MgSO₄. One or more additional salts such as potassium chloride (KCl), calcium chloride (CaCl) and ammonium sulfate [(NH₄)₂SO₄] may also be present in the reaction mixture. The salts can optionally also be included in the elution/lysis buffer as needed.

In certain embodiments, the RT-LAMP or LAMP takes place at a constant temperature of 60° C., 61° C., 62° C., 63° C., 64° C., 65° C., 66° C., 67° C., 68 or 69° C. in a reaction mixture comprising at least one set of 4-6 oligonucleotide primers directed to a target nucleic acid of an organism, and optionally comprising a second set of 4-6 oligonucleotide primers directed to a target human nucleic acid, such as beta actin, deoxyribonucleotide triphosphates (dNTPs) including dUTP, a source of magnesium ions (e.g., MgCl₂ or MgSO₄), one or more reagents for detection of the amplified target nucleic acid, which may include at least one set of DARQ primers and a fluorescent DNA intercalating agent, and one or more enzymes selected from a reverse transcriptase, a DNA polymerase, and a dual specificity reverse transcriptase/DNA polymerase enzyme. The reaction mixture may also contain one or more of an RNase inhibitor, a serum albumin, a reducing agent such as tris(2-carboxyethyl)phosphine (TCEP) and salts thereof, e.g., TCEP-HCl, thermolabile uracil-DNA glycosylase (UDG), a buffering agent, salts such as (NH4)₂SO₄ and KCl, and a non-ionic surfactant. In embodiments, the non-ionic surfactant may be selected from Triton™ X-100 or similar Triton™ detergent, or a Tween™ detergent such as Tween 20 or Tween 80, or a maltoside such as n-dodecyl-β-D-maltoside. In embodiments, the reaction mixture is lyophilized and may further contain one or more of dextran, mannitol, sorbitol, maltodextrin, trehalose, lactose, and/or lactitol. In embodiments, the lyophilized reaction mixture comprises trehalose, lactose and/or lactitol.

As used herein, the term “target nucleic acid” refers to a nucleic acid of an organism, or a part (e.g., a gene) of a nucleic acid of an organism that is targeted to be amplified by an isothermal nucleic acid amplification reaction described herein. In certain aspects, the target nucleic acid comprises one or more RNA or DNA molecules of an organism. In other aspects, the target nucleic acid comprises one or more genes of an organism. As used herein, the term “organism” includes microorganisms including, but not limited to, bacteria, viruses, fungi, algae, protozoa, and prion genes, as well as organisms such as parasites, which include, but are not limited to, certain kinds of worms (e.g., hookworms, ringworms), plants, and insect larvae.

In certain embodiments, the target nucleic acid comprises one or more RNA or DNA molecules of an organism selected from a virus, a bacterium, a fungus or a parasite. In specific embodiments, the organism is a virus. In certain instances, the virus includes, but is not limited to, rhinovirus, adenovirus, influenza virus, respiratory syncytial virus, enterovirus D68, hepatitis C virus (HCV), West Nile virus, Zika virus, Sindbis virus (SINV), dengue virus, Ebola virus, Marburg virus, Crimean-Congo hemorrhagic fever (CCHF) orthonairovirus (CCHFV), yellow fever virus, Rift Valley fever virus (RVFV), Omsk hemorrhagic fever virus (OHFV), Kyasanur Forest disease virus (KFDV), Junin virus, Machupo virus, Sabia virus, Guanarito virus, Garissa virus, Ilesha virus, Lassa fever virus, human coronavirus (HCoV)-HKU-1, HCoV-NL63, HCoV-0C43 and HCoV-229E, severe acute respiratory syndrome coronavirus (SARS-Cov), Middle East respiratory syndrome coronavirus (MERS-CoV), and severe acute respiratory syndrome coronavirus 2 (SARS-CoV-2). In certain embodiments, the virus is selected from SARS-Cov, MERS-CoV, and SARS-CoV-2. In specific embodiments, the virus is SARS-CoV-2.

As used herein, a “subject” refers to any mammal infected or suspected of being infected or at risk of being infected with an organism (e.g., a microorganism such as SARS-CoV-2). The methods of the present disclosure are generally written as applicable to human subjects, but the methods may be applied to other mammalian subjects. Accordingly, in certain embodiments a method described herein may be performed on a “subject” which may include any mammal, for example a human, primate, mouse, rat, dog, cat, cow, horse, goat, camel, sheep or a pig. In specific embodiments, a subject or a subject in need is a human, such as a human infant, child, adolescent or adult.

In embodiments, the methods further comprise adding one or more RNase inhibitors to the RT-LAMP or LAMP reaction mixture. In embodiments, the RNase inhibitor is optionally present in the lysis buffer where the method comprises a chemical lysis step. In some embodiments, the RNase inhibitor is added to the sample prior to performing the clarification step and prior to any thermal treatment step. The term “inhibitor” refers to an agent (e.g., a chemical or biological molecule) that produces an alteration, interference, reduction, down regulation, blocking, abrogation or degradation, directly or indirectly, in the expression, amount or activity of a target molecule, wherein the alteration, interference, reduction, down regulation, blocking, abrogation or degradation is statistically, biologically, or clinically significant. The term “RNase inhibitor” refers to an agent that may block, inactivate, reduce or minimize the activity of an RNase enzyme, which normally functions to fragment RNA molecules. RNase inhibitors are commonly used as a precautionary measure in enzymatic manipulations of RNA, such as in the methods described herein, to inhibit and control for RNase contaminants in the sample. In some embodiments, the RNase inhibitor remains active at elevated temperatures of from about 60° C. to about 100° C., preferably about 65° C. to about 95° C., most preferably about 95° C.

Exemplary RNase inhibitors that may be used in the assay methods described here include, without limitations, porcine liver RNase inhibitors, human placental RNase inhibitors, murine RNase inhibitors, rat lung RNase inhibitors, and rat liver RNase inhibitors. Exemplary RNase inhibitors include, without limitations, Riboprotect Hu, RiboGuard™, RNasin™ or RNasin™ Plus, Ribolock, RiboShield, RNaseOUT™, or Superase In™. In embodiments, the final concentration of RNase inhibitor is from about 0.01 U/μl to 2.0 U/μl, preferably about 0.5 U/μl to about 2.0 U/μl. In some embodiments, the RNase inhibitor is polyvinylsulfonic acid (PVSA).

In certain embodiments, the methods described here allow for rapid detection of the amplified target nucleic acid. As used herein, the term “rapid detection” indicates that the isothermal nucleic acid amplification reaction amplifies a target nucleic acid sufficiently to be detected quantitatively and/or qualitatively by one or more procedures known in the art (e.g., fluorescence and/or colorimetric detection) within a period of about 1 minute to about 60 minutes, preferably about 5 minutes to about 45 minutes, or from about 5 minutes to 30 minutes, or more preferably about 10 minutes to about 30 minutes, or from about 10 minutes to about 26 minutes, or from about 15 minutes to about 30 minutes. Preferably, according to the methods described here, the isothermal nucleic acid amplification reaction amplifies a target nucleic acid sufficiently to be detected within a period of from 5 to 26 minutes.

Thus, in certain embodiments, the methods allow for rapid detection of the amplified target nucleic acid within a period of about 5 to 30 minutes, or about 10 to 30 minutes, or from about 10 to about 26 minutes, or 10 to 20 minutes, or from about 15 to about 30 minutes, or from about 15 minutes to 20 minutes. In additional embodiments, the total time required to perform the methods described herein is from about 5 minutes to about 60 minutes, or about 25 minutes to about 60 minutes, or from about 30 minutes to about 60 minutes.

In some embodiments, the methods described here may further comprise one or more additional steps performed prior to the isothermal nucleic acid amplification reaction. The one or more additional steps may include one or more of a clarification step comprising simultaneous ultrafiltration and concentration, chemical lysis, and thermal treatment.

As used herein, the term “chemical lysis” refers to a process of permeabilizing the cell membrane of an organism (or the outer protein coat of a virus) to facilitate release of genetic material (e.g., RNA or DNA) from said organism. Examples of suitable lysis agents include, but are not limited to, bioactive reagents such as lysis enzymes that are used for lysis of different cell types, e.g., gram positive or negative bacteria, plants, yeast, mammalian, such as lysozymes, achromopeptidase, lysostaphin, labiase, kitalase, lyticase, and a variety of other commercially available lysis enzymes. In embodiments, the lysis reagent is selected from a non-ionic surfactant, guanidine hydrochloride (GuHCl) and guanidine thiocyanate (GuSCN). In embodiments, the lysis reagent is a non-ionic surfactant. In embodiments, the non-ionic surfactant is selected from Triton™ X-100 or similar Triton™ detergent, or a Tween™ detergent such as Tween 20 or Tween 80, or a maltoside such as n-dodecyl-β-D-maltoside.

Other lysis agents can additionally or alternatively be added to the sample to facilitate permeabilization of an organism present in the sample. For example, surfactant-based lysis solutions can be used to lyse the organism. Lysis solutions can include ionic surfactants such as, for example, sarcosyl and sodium dodecyl sulfate (SDS) or non-ionic surfactants such as Triton™ X 100 or other similar Triton™ detergents, or a Tween™ detergent such as Tween 20 or Tween 80, or a maltoside such as n-dodecyl-β-D-maltoside. More generally, chemical lysis agents can include, without limitation, organic solvents, chelating agents, detergents, surfactants, and chaotropic agents.

In an embodiment, chemical lysis comprises contacting the sample with a lysis reagent selected from a non-ionic surfactant, guanidine hydrochloride (GuHCl) and guanidine thiocyanate (GuSCN) in a buffered solution. In embodiments, the lysis reagent is a non-ionic surfactant. In embodiments, the non-ionic surfactant is selected from Triton™ X-100 or similar Triton™ detergent, or a Tween™ detergent such as Tween 20 or Tween 80, or a maltoside such as n-dodecyl-β-D-maltoside. In embodiments, the buffered solution further comprises sodium dodecyl sulfate (SDS).

In embodiments, the organism can be permeabilized by non-chemical permeabilization methods. Exemplary non-chemical permeabilization methods that can be used include, but are not limited to, physical lysis techniques such as electroporation, mechanical permeabilization methods (e.g., bead beating using a homogenizer and grinding balls to mechanically disrupt sample tissue structures), acoustic permeabilization (e.g., sonication), and thermal lysis techniques such as heating to induce thermal permeabilization of the organism.

In embodiments, a medium, solution, or permeabilization solution may contain one or more proteases. In some embodiments, a biological sample treated with a protease capable of degrading histone proteins can result in the generation of fragmented genomic DNA.

As used herein, the term “thermal treatment” refers to a process of applying a source of heat to a sample that results in an increase in the temperature of said sample. In certain embodiments, the thermal treatment results in an increase in the temperature of the sample by from about 20° C. to about 50° C., preferably about 20° C. to about 40° C., most preferably about 30° C. to about 40° C. In certain embodiments, subjecting the sample to thermal treatment comprises heating the sample to a temperature of from about 80° C. to about 100° C., preferably about 90° C. to about 98° C., most preferably about 95° C., and for a period of time from about 30 seconds to about 10 minutes, preferably about 30 seconds to about 5 minutes, most preferably from about 1 minute to about 3 minutes. In specific embodiments, subjecting the sample to thermal treatment comprises heating the sample to 95° C. for 3 minutes.

In embodiments, the methods may comprise a clarification step comprising simultaneous ultrafiltration and concentration to produce a clarified sample performed prior to the isothermal nucleic acid amplification reaction. As used herein, the term “clarification” refers to a process that removes cellular proteins and lipids, cellular debris, and other contaminating particulates from a sample, but does not remove the analyte to be detected, to produce a “clarified” sample. In certain embodiments, clarification of a sample comprises one or more steps including filtration, ultrafiltration, centrifugation, ultracentrifugation, and sonication of the sample. The steps may be simultaneous or separate. The term “simultaneous” refers to two or more steps (e.g., clarification steps) that occur at the same time.

The term “ultrafiltration” refers to a variety of membrane filtration devices in which forces like pressure or concentration gradients lead to a separation through a semipermeable membrane. Ultrafiltration membranes are defined by the molecular weight cut-off (MWCO) of the membrane used. Exemplary ultrafiltration units include, but are not limited to, an Amicon or Millipore ultrafiltration unit. In certain aspects, the filter is selected from the group consisting of a copolymer styrene filter and a butadiene filter. In specific embodiments, the membrane is a cellulose membrane. In certain embodiments, the membrane has a molecular weight cut-off (MWCO) of from about 1 kDa to about 300 kDa, preferably about 3 kDa to about 200 kDa, most preferably about 100 kDa to about 200 kDa.

In embodiments, the clarification step comprises centrifugation of the sample through a filter having a membrane that does not allow the analyte, e.g., viral RNA or virus particles, to pass through the filter, while other molecules and impurities pass through. In some embodiments, the centrifugation is performed at a speed of from about 10,000×g to 150,000×g, 10,000 to 100,000×g, 10,000 to 20,000×g, or from 14,000 to 15,000×g, and for a period of time from about 5 minutes to 30 minutes, preferably 10 minutes to 20 minutes, most preferably about 20 minutes. In specific embodiments, the centrifugation is performed at a speed of 10,000-20,000×g for 20 minutes.

In certain embodiments, by the end of the simultaneous ultrafiltration and concentration of the sample, the volume of the clarified sample left in the filter is from about 1 μl to about 25 μl, preferably from 5 μl to about 20 μl, most preferably about 10 μl to about 15 μl. In certain aspects, the simultaneous ultrafiltration and concentration step reduces the volume of the sample by from about 90% to about 99%, more preferably about 95% to about 98%, most preferably about 97% to about 98%. In specific embodiment, the simultaneous ultrafiltration and concentration step reduces the volume of the sample from about 500 μl to about 15-20 μl.

In some embodiments, the clarification step is performed prior to performing to chemical lysis and/or thermal treatment. It was unexpectedly found that performing the clarification step prior to performing the one or more additional steps selected from the group consisting of subjecting the sample to chemical lysis and subjecting the sample to thermal treatment led to a marked improvement in sensitivity for some embodiments of the methods described herein. In certain aspects, performing the clarification step prior to performing the one or more additional steps selected from the group consisting of subjecting the sample to chemical lysis and subjecting the sample to thermal treatment reduced the LOD of the described methods by about 2 fold to about 20 fold, preferably about 4 fold to about 10 fold, compared to when the method was carried out without the clarification step, or when the clarification step was performed after performing the one or more additional steps selected from the group consisting of subjecting the sample to chemical lysis and subjecting the sample to thermal treatment.

In certain embodiments, the methods may further comprise dividing the clarified sample into 2, 3, 4, 5, 6, 7, 8, 9, or 10 portions prior to conducting the isothermal nucleic acid amplification reaction. In some aspects, the methods further comprise subjecting the second, third, fourth, fifth, sixth, seventh, eighth, ninth, and/or tenth portion of the clarified sample to an isothermal nucleic acid amplification reaction to amplify a target nucleic acid, and detecting the amplified target nucleic acid. In certain embodiments, a different target nucleic acid is amplified in each portion of the clarified sample. In other embodiments, the same target nucleic acid is amplified in at least two portions of the clarified sample. Thus, in some embodiments, the methods further comprise subjecting a second portion of the clarified sample to an isothermal nucleic acid amplification reaction to amplify a target nucleic acid, and detecting the amplified target nucleic acid. In certain aspects, the target nucleic acid amplified in the first portion of the sample and the target nucleic acid amplified in the second portion of the sample are the same. In other aspects, the target nucleic acid amplified in the first portion of the sample and the target nucleic acid amplified in the second portion of the sample are different. In yet other embodiments, the methods further comprise subjecting a third portion of the clarified sample to an isothermal nucleic acid amplification reaction to amplify a third target nucleic acid, and detecting the amplified target nucleic acid.

In certain aspects, the target nucleic acid amplified in the first portion of the sample and the target nucleic acid amplified in the third portion of the sample are the same. In other aspects, the target nucleic acid amplified in the first portion of the sample and the target nucleic acid amplified in the third portion of the sample are different. In additional aspects, the target nucleic acid amplified in the second portion of the sample and the target nucleic acid amplified in the third portion of the sample are the same. In other aspects, the target nucleic acid amplified in the second portion of the sample and the target nucleic acid amplified in the third portion of the sample are different. In further aspects, the target nucleic acid amplified in the first portion of the sample and the target nucleic acid amplified in the second and third portions of the sample are the same. In additional aspects, the target nucleic acid amplified in the first portion of the sample and the target nucleic acid amplified in the second and third portions of the sample are different.

### Kits

The disclosure also provides kits for detection of an organism in a sample. In embodiments, the disclosure provides kits for detection of a SARS-CoV-2 virus. In embodiments, the kit comprises a first container comprising a buffer for sample elution and lysis and optionally housing a centrifugal filter assembly comprising a filter having a membrane that does not allow the SARS-CoV-2 virus to pass through, a second container comprising a set of 4-6 lyophilized oligonucleotide primers selected from any one of primer sets identified by SEQ ID NOs 1-6 (Set 1), SEQ ID NOs 7-12 (Set 2), SEQ ID NOs 7, 13, 9, 14, 11, and 15 (Set 3), or SEQ ID NOs 7-12 and SEQ ID NOs 16 and 17 (Set 4), and reagents for performing a reverse transcription and loop-mediated isothermal nucleic acid amplification reaction (RT-LAMP). In embodiments, the membrane has a molecular weight cut-off (MWCO) of from about 1 kDa to about 300 kDa, preferably about 3 kDa to about 200 kDa, more preferably about 100 kDa to about 200 kDa. In embodiments, the reagents for performing RT-LAMP comprise deoxyribonucleotide triphosphates (dNTPs), one or more reagents for detection of the amplified target nucleic acids, a reverse transcriptase, and a DNA polymerase. In embodiments, one or more of the reagents for performing RT-LAMP is lyophilized. In embodiments, the lyophilized reagents comprise dNTPs and a buffer. In embodiments, the kit further comprises one or more of an RNase inhibitor, a positive control sample, a negative control sample, and one or more fluorescent or colorimetric indicators for detection of an amplified target nucleic acid.

In certain aspects, the filter is selected from the group consisting of a copolymer styrene filter and a butadiene filter. In specific embodiments, the membrane is a cellulose membrane. In certain embodiments, the membrane has a molecular weight cut-off (MWCO) of from about 1 kDa to about 300 kDa, preferably about 3 kDa to about 200 kDa, most preferably about 100 kDa to about 200 kDa. In other aspects, the membrane has a pore size of from about 0.001 to about 0.1 microns, preferably about 0.001 to about 0.004 microns, most preferably 0.001 to about 0.002 microns.

In some embodiments, the kits further comprise one or more positive control and negative control samples. In other embodiments, the kits further comprise one or more fluorescent or colorimetric indicators for detection of an amplified target nucleic acid.

In certain embodiments, the kits further comprise reagents for performing RT-LAMP comprising a buffering agent, salts such as (NH4)₂SO₄ and KCl, a mixture of deoxyribonucleotide triphosphates (dNTPs), one or more reagents for detection of the amplified target nucleic acids, a source of magnesium ions such as magnesium chloride (MgCl₂) or magnesium sulfate (MgSO₄), a reverse transcriptase, and a DNA polymerase. In some embodiments, one or more of the reagents for performing RT-LAMP is lyophilized. The term “lyophilized” refers to a water removal process typically used to preserve perishable materials (e.g., enzymes), to extend shelf life or make the material more convenient for transport. Lyophilization works by freezing the material, then reducing the pressure and adding heat to allow the frozen water in the material to sublimate. This is in contrast to dehydration by most conventional methods that evaporate water using heat.

In embodiments, the lyophilized reagents comprise a surfactant, a buffering agent, salts such as (NH4)₂SO₄ and KCl, a source of magnesium ions such as magnesium chloride (MgCl₂) or magnesium sulfate (MgSO₄), a reverse transcriptase, a DNA polymerase, a mixture of dNTPs, at least one primer set for the amplification of a target nucleic acid sequence, preferably including 4-6 RT-LAMP or LAMP primers and a DARQ primer set, and a fluorescent DNA intercalating dye.

In embodiments, the surfactant is a non-ionic surfactant, such as Triton X-100 or similar Triton™ detergent, or a Tween™ detergent such as Tween 20 or Tween 80, or a maltoside such as n-dodecyl-β-D-maltoside.

In embodiments, the buffering agent is Tris, CAPS, HEPES, MOPS, PIPES, TAPS, TE, TES, or Tricine. In embodiments, the RNase inhibitor is polyvinylsulfonic Acid (PVSA).

In embodiments, the magnesium sulfate is present in an amount suitable to provide a concentration of 2-20 mM, or 4-10 mM in the reconstituted solution.

In embodiments, the reverse transcriptase has reduced RNase H activity.

In embodiments, the DNA polymerase is a thermostable DNA polymerase. In embodiments, the DNA polymerase is selected from a Geobacillus bogazici DNA polymerase, a Bst DNA polymerase, or a Taq DNA polymerase.

In embodiments, the mixture of dNTPs includes dATP, dCTP, dGTP, dTTP, and dUTP, each independently present in a range of from about 0.28-7 mM or 0.7-2.8 mM in the reconstituted solution.

In embodiments, the at least one primer set is a primer set described in Table 1. In embodiments, the lyophilized reagents comprise a second primer set for amplification of a control sequence, such as human actin. Preferably the reaction mixture comprises at least one DARQ primer set.

In embodiments, the fluorescent DNA intercalating dye is SYBR green or Chai Green™, or similar dye such as SYBR Green I and II, SYBR Safe, SYBR Gold, Eva Green, Ethidium Bromide, Oxazole yellow-based cyanine dyes (e.g. YOYO-1, DiYO-1, TOTO-1, DiTO-1, TOTO-3), Pico Green, SYTO 9, SYTO 13, SYTO 16, SYTO 60, SYTO 62, SYTO 64, SYTO 82, Boxto, Miami Green, Miami Yellow, Miami Orange.

In embodiments, the lyophilized reagents further comprise one or more of a serum albumin, such as bovine serum albumin (BSA), fetal bovine serum, or human serum albumin, for example up to 1 mg/ml serum albumin in the reconstituted solution; a reducing agent such as tris(2-carboxyethyl)phosphine (TCEP) and salts thereof, e.g., TCEP-HCl, for example up to 4 mM in the reconstituted solution; one or more of trehalose, lactose, and lactitol, from about 5-50% w/v or from about 10-45% w/v, or from 10-20% w/v in the reconstituted solution; thermolabile Uracil-DNA Glycosylase (UDG); and an RNase inhibitor, such as Riboprotect Hu (Blirt), RiboGuard™ (Lucigen), RNasin™ or RNasin™ Plus (Promega), RNaseOUT™, Superase In™, RiboLock (Thermo Scientific), RiboShield (PCR Biosystems). In embodiments, the lyophilized reagents may also comprise one or more of dextran, mannitol, sorbitol, and/or maltodextrin.

In an embodiment, the kit comprises a collection tube (A), a sterile swab (B), and a test pouch (C). The test pouch contains a transfer pipette (C1) and a tube assembly (C2). The collection tube (A) contains a buffer for sample elution and lysis. In embodiments, the buffer comprises a surfactant, preferably a non-ionic surfactant, such as Triton™ X-100 or similar Triton™ detergent, or a Tween™ detergent such as Tween 20 or Tween 80, or a maltoside such as n-dodecyl-β-D-maltoside; a buffering agent, such as Tris, CAPS, HEPES, MOPS, PIPES, TAPS, TE, TES, or Tricine; an RNase inhibitor, such as polyvinylsulfonic acid (PVSA); and nuclease-free water. The tube assembly (C2) contains a lyophilized mixture of reagents for performing RT-LAMP and a magnetic bead, such as a stainless steel ball bearing, used to mix the solution inside the test tube during the RT-LAMP reaction. For example, the reagents may include a suitable buffer, such as a Tris buffer; a non-ionic surfactant, such as Triton™ X-100 or similar Triton™ detergent, or a Tween™ detergent such as Tween 20 or Tween 80, or a maltoside such as n-dodecyl-β-D-maltoside; salts such as (NH4)₂SO₄ and KCl, magnesium sulfate to provide a concentration of 2-20 mM, or 4-10 mM in the reconstituted solution; a mixture of deoxy nucleotides (dNTPs), for example including dATP, dCTP, dGTP, dTTP, and dUTP, each independently present in a range of from about 0.28-7 mM or 0.7-2.8 mM in the reconstituted solution; at least one primer set for the amplification of target sequences, e.g., target viral sequences such as a primer set described herein; an optional second primer set for amplification of a control sequence, such as human beta-actin; one or more sets of detection primers; a dye suitable for detection of amplified DNA, for example a fluorescent DNA intercalating dye such as a SYBR green or Chai Green™, or similar dye such as SYBR Green I and II, SYBR Safe, SYBR Gold, Eva Green, Ethidium Bromide, Oxazole yellow-based cyanine dyes (e.g. YOYO-1, DiYO-1, TOTO-1, DiTO-1, TOTO-3), Pico Green, SYTO 9, SYTO 13, SYTO 16, SYTO 60, SYTO 62, SYTO 64, SYTO 82, Boxto, Miami Green, Miami Yellow, Miami Orange; a DNA polymerase, preferably a thermostable DNA polymerase, such as a Geobacillus bogazici DNA polymerase, a Bst DNA polymerase (e.g., BST 2.0), or a Taq DNA polymerase; and a reverse transcriptase, preferably one with reduced RNase H activity. Additional reagents may also be present, for example one or more of a serum albumin, such as bovine serum albumin (BSA), fetal bovine serum, or human serum albumin, for example up to 1 mg/ml serum albumin in the reconstituted solution; a reducing agent such as TCEP, for example up to 4 mM in the reconstituted solution; one or more of trehalose, lactose, and lactitol, from about 5-50% w/v or from about 10-45% w/v, or from 10-20% w/v in the reconstituted solution; thermolabile UDG; and an RNase inhibitor, such as Riboprotect Hu (Blirt), RiboGuard™ (Lucigen), RNasin™ or RNasin™ Plus (Promega), RNaseOUT™, Superase In™, RiboLock (Thermo Scientific), RiboShield (PCR Biosystems). In embodiments, the lyophilized reagents may also comprise one or more of dextran, mannitol, sorbitol, and/or maltodextrin.

In some embodiments, the invention provides methods for detecting an organism in a sample, the methods comprising subjecting the sample to a clarification step comprising simultaneous ultrafiltration and concentration to produce a clarified sample, followed by subjecting a first portion of the clarified sample to a first isothermal nucleic acid amplification reaction to amplify a first target nucleic acid of the organism, and detecting amplified target nucleic acid, wherein detection of the amplified target nucleic acid indicates presence of the organism in the sample.

In an embodiment, the method is performed within a period of time from 20 to 120 minutes, preferably 20 to 90 minutes, most preferably 20 to 40 minutes. In certain embodiments, the methods are performed within a period of time from 5 to 45 minutes, preferably 10 to 30 minutes, most preferably 15 to 20 minutes.

In an embodiment, the method further comprises subjecting a second portion of the clarified sample to a second isothermal nucleic acid amplification reaction to amplify a second target nucleic acid, and detecting the amplified target nucleic acid. In an embodiment, the first and second target nucleic acids are the same or different. In an embodiment, two different sets of primers are used to amplify the first and second target nucleic acids.

In an embodiment, the method further comprises one or more additional steps prior to performing the isothermal nucleic acid amplification reaction, the one or more additional steps selected from the group consisting of subjecting the sample to chemical lysis and subjecting the sample to thermal treatment.

In an embodiment, the subjecting the sample to chemical lysis comprises contacting the sample with a lysing reagent selected from non-ionic surfactant, guanidine hydrochloride and guanidine thiocyanate.

In an embodiment, the subjecting the sample to thermal treatment comprises heating the sample to about 95° C. for a period of time from 30 seconds to 10 minutes, preferably 30 seconds to 5 minutes, most preferably 1 minute to 3 minutes. In an embodiment, the subjecting the sample to thermal treatment comprises heating the sample to 95° C. for 3 minutes.

In an embodiment, an RNase inhibitor is added to the sample prior to performing the clarification step.

In an embodiment, the clarification step is performed by a method comprising centrifugation. In an embodiment, the clarification step is performed by a method comprising ultrafiltration through a filter comprising a membrane having a molecular weight cut-off (MWCO) of from about 1 kDa to 300 kDa, preferably 3 kDa to 200 kDa, most preferably 100 kDa to 200 kDa. In an embodiment, the filter is selected from a copolymer styrene filter and a butadiene filter. In an embodiment, the membrane is a cellulose membrane. In an embodiment, the centrifugation is performed at a speed of from about 14,000 to 15,000×g for about 10 to 20 minutes.

In an embodiment of any of the foregoing methods, the isothermal nucleic acid amplification is a loop-mediated isothermal nucleic acid amplification reaction (LAMP). In an embodiment, the target nucleic acid is an RNA and the LAMP further comprises reverse transcription (RT-LAMP). In an embodiment, the RT-LAMP or LAMP takes place at a constant temperature of 65 or 67° C. in a reaction mixture comprising a set of six oligonucleotide primers, deoxyribonucleotide triphosphates (dNTPs), a source of magnesium ions (e.g., MgCl₂ or MgSO₄), one or more reagents for detection of the amplified target nucleic acid, and one or more enzymes selected from a reverse transcriptase, a DNA polymerase, and a dual specificity reverse transcriptase/DNA polymerase enzyme.

In an embodiment, the one or more reagents for detection of the amplified target nucleic acid comprise one or more fluorescent DNA indicator molecules. In an embodiment, the one or more fluorescent DNA indicator molecules is selected from the group consisting of fluorescein isothiocyanate (FITC), rhodamine X (ROX), 6-carboxyfluorescein (FAM), SYTO 9, SYTO 82, SYTO 16, SYTO 13, SYTO 64, Boxto, Miami Green, Miami Yellow, Miami Orange, YOPRO 1, SYTO 62, TOPRO 3, SYTO 60, EvaGreen, Chai Green™, POPO 3, NG-DCS 1, SYBR Green I, SYBR Green II, BOBO 3, TOTO 3, Pico 488, TOTO 1, and SYTO 24. In an embodiment, the detection of the amplified target nucleic acid comprises detecting an increase in a fluorescent signal in the sample over a period of time. In an embodiment, the one or more reagents for detection of the amplified nucleic acid comprises one or more colorimetric indicators selected from the group consisting of malachite green, calcein and hydroxynaphthol blue. In an embodiment, the one or more reagents for detection of the amplified nucleic acid comprises a pH-sensitive colorimetric indicator selected from the group consisting of phenol red, cresol red, neutral red, bromocresol purple, and m-cresol purple.

In an embodiment of any of the foregoing methods, the detection of the target nucleic acid in the sample has a limit of detection (LOD) of from 1 to 100 copies, preferably 1 to 5 copies of the target nucleic acid per microliter (μl). In an embodiment, the detection of the target nucleic acid in the sample has a limit of detection (LOD) of 1 copy of the target nucleic acid per microliter (μl). In an embodiment, the method has a sensitivity of from about 90% to about 100%, preferably from about 95% to about 100%, most preferably at least about 97%. In an embodiment, the method has a specificity of from about 90% to about 100%, preferably from about 95% to about 100%, most preferably at least about 99%.

In an embodiment of any of the foregoing methods, the target nucleic acid comprises one or more RNA or DNA molecules of an organism selected from a virus, a bacterium, a fungus or a parasite. In an embodiment, the target nucleic acid is of a virus. In embodiments, the virus is selected from severe acute respiratory syndrome coronavirus (SARS-CoV), Middle East respiratory syndrome coronavirus (MERS-CoV), and severe acute respiratory syndrome coronavirus 2 (SARS-CoV-2). In embodiments, the virus is SARS-Cov-2.

In an embodiment of the method where the virus is SARS-CoV-2, the set of 4-6 oligonucleotide primers is selected from the group consisting of Set 1, Set 2, Set 3, and Set 4, identified by the sequence identifiers set forth in Table 1, absent the LoopF and LoopB primers, i.e., SEQ ID NOs 1-4 of Set 1, SEQ ID NOs 7-10 of Set 2, SEQ ID NOs 7, 13, 9, and 14, of Set 3, or SEQ ID NOs 7-10 and SEQ ID NOs 16 and 17 of Set 4. In embodiments, the set of oligonucleotide primers hybridizes to gene N or gene M of SARS-CoV-2 viral RNA. In an embodiment of the method where the virus is SARS-CoV-2, the set of oligonucleotide primers is selected from the group consisting of Set 1, Set 2, Set 3, and Set 4, identified by the sequence identifiers set forth in Table 1, i.e., SEQ ID NOs 1-6 (Set 1), SEQ ID NOs 7-12 (Set 2), SEQ ID NOs 7, 13, 9, 14, 11, and 15 (Set 3), or SEQ ID NOs 7-12 and SEQ ID NOs 16 and 17 (Set 4). In embodiments, the set of oligonucleotide primers hybridizes to gene N or gene M of SARS-CoV-2 viral RNA.

In an embodiment of the method where the virus is SARS-Cov-2, the set of oligonucleotide primers is set 4 identified in Table 1, SEQ ID NOs 7-12 and SEQ ID NOs 16 and 17.

In an embodiment of any of the foregoing methods, the sample is a biological sample or an environmental sample. In embodiments, the biological sample comprises saliva, mucus, blood, or serum. In embodiments, the environmental sample comprises particles collected from a solid surface, a liquid, or a volume of air. In embodiments, the biological sample is an upper respiratory tract sample. In embodiments, the upper respiratory tract sample is obtained from a nasopharyngeal (NP) swab, an oropharyngeal (OP) swab, a nasal swab, a nasal aspirate, or a nasal wash, further optionally wherein the nasal swab is an anterior nasal swab or a mid-turbinate nasal swab.

The disclosure provides a method for detecting a SARS-CoV-2 virus in a biological sample obtained from a subject, the method comprising subjecting the sample to a clarification step comprising simultaneous ultrafiltration and concentration to produce a clarified sample, followed by subjecting a first portion of the clarified sample to a first reverse transcription and loop-mediated isothermal nucleic acid amplification reaction (RT-LAMP) to amplify a first target nucleic acid of the SARS-CoV-2 virus selected from gene M and gene N, and detecting the amplified target nucleic acid, wherein detection of the amplified target nucleic acid indicates presence of the SARS-CoV-2 virus in the biological sample. In embodiments, the biological sample is a saliva or mucous sample of a human subject. In embodiments, the method further comprises subjecting a second portion of the clarified sample to a second isothermal nucleic acid amplification reaction to amplify a second target nucleic acid, and detecting the amplified second target nucleic acid. In embodiments, the first and second target nucleic acids are the same or different. In embodiments, two different sets of primers are used to amplify the first and second target nucleic acids. In embodiments, the RT-LAMP reaction takes place at a constant temperature of 65° C. or 67° C. in a reaction mixture comprising a set of six oligonucleotide primers, deoxyribonucleotide triphosphates (dNTPs), a source of magnesium ions (e.g., MgCl₂ or MgSO₄), one or more reagents for detecting the amplified target nucleic acid, a reverse transcriptase, and a DNA polymerase. In embodiments, the method further comprises one or more additional steps prior to performing the isothermal nucleic acid amplification, as discussed above.

In embodiments, the set of four or six oligonucleotide primers hybridize to gene N or gene M of SARS-CoV-2 viral RNA. In embodiments, the set of four or six oligonucleotide primers is selected from the group consisting of Set 1, Set 2, Set 3, and Set 4, identified by SEQ ID NOs 1-6 (Set 1), SEQ ID NOs 7-12 (Set 2), SEQ ID NOs 7, 13, 9, 14, 11, and 15 (Set 3), and SEQ ID NOs 7-12 and SEQ ID NOs 16 and 17 (Set 4). In embodiments, each primer in the set is labeled with one or more colorimetric indicators. In embodiments, the one or more reagents for detection of the amplified target nucleic acid comprise one or more fluorescence DNA indicators selected from the group consisting of fluorescein isothiocyanate (FITC), Rhodamine X (ROX), Hexachlorofluorescein (HEX), 6-carboxyfluorescein (FAM), SYTO 9, SYTO 82, SYTO 16, SYTO 13, SYTO 64, Boxto, Miami Green, Miami Yellow, Miami Orange, YOPRO 1, SYTO 62, TOPRO 3, SYTO 60, Chai Green™, EvaGreen, POPO 3, NG-DCS 1, SYBR Green I, SYBR Green II, BOBO 3, TOTO 3, Pico 488, TOTO 1, and SYTO 24.

In certain embodiments, the disclosure provides methods for rapid detection of SARS-CoV-2 in a biological sample obtained from a subject, the method comprising: i) treating the biological sample with an RNase inhibitor; ii) subjecting the biological sample to a clarification step comprising simultaneous ultrafiltration and concentration to produce a clarified sample; iii) subjecting a first portion of the clarified sample to chemical lysis comprising treating the sample with a lysis buffer followed by thermal treatment comprising heating the sample to 95° C. for 3 minutes; iv) subjecting the lysed sample to a reverse transcription and loop-mediated isothermal nucleic acid amplification reaction (RT-LAMP) to amplify a first target nucleic acid selected from the group consisting of the N and M genes of SARS-CoV-2 viral RNA, wherein the RT-LAMP takes place at a constant temperature of 65° C. or 67° C. in a reaction mixture comprising a set of four or six oligonucleotide primers, deoxyribonucleotide triphosphates (dNTPs), a source of magnesium ions (e.g., MgCl₂ or MgSO₄), one or more reagents for detecting the amplified target nucleic acid, a reverse transcriptase, and a DNA polymerase; and v) detecting the amplified target nucleic acid using one or more fluorescent or colorimetric indicators, wherein the isothermal nucleic acid amplification reaction allows detection of the amplified target nucleic acid occurs within about 10 to about 20 g wherein detection of the amplified target nucleic acid indicates presence of SARS-CoV-2 in the biological sample.

In some embodiments, the methods for rapid detection of SARS-CoV-2 may further comprise subjecting a second portion of the clarified sample to a second isothermal nucleic acid amplification reaction to amplify a second target nucleic acid, and detecting the amplified second target nucleic acid. In other embodiments, the methods for rapid detection of SARS-CoV-2 may further comprise subjecting a third portion of the clarified sample to a third isothermal nucleic acid amplification reaction to amplify a third target nucleic acid, and detecting the amplified third target nucleic acid.

In additional embodiments, the disclosure provides methods for detecting SARS-CoV-2 in a biological sample obtained from a subject, the method comprising subjecting the sample to a clarification step comprising simultaneous ultrafiltration and concentration to produce a clarified sample, followed by subjecting a first portion of the clarified sample to a reverse transcription and loop-mediated isothermal nucleic acid amplification reaction (RT-LAMP) to amplify a target nucleic acid selected from the group consisting of the N and M genes of SARS-CoV-2 viral RNA, and detecting the amplified target nucleic acid, wherein detection of the amplified target nucleic acid indicates presence of SARS-CoV-2 in the biological sample.

In accordance with embodiments of any of the methods described here, the set of four or six oligonucleotide primers is selected from the group consisting of Set 1, Set 2, Set 3, and Set 4, identified by SEQ ID NOs 1-6 (Set 1), SEQ ID NOs 7-12 (Set 2), SEQ ID NOs 7, 13, 9, 14, 11, and 15 (Set 3), and SEQ ID NOs 7-12, 16 and 17 (Set 4). In embodiments, the method further comprises subjecting a second portion of the clarified sample to a second isothermal nucleic acid amplification reaction to amplify a second target nucleic acid, and detecting the amplified second target nucleic acid. In embodiments, the first and second isothermal nucleic acid amplification reactions are performed with a different set of primers selected from the group consisting of Set 1, Set 2, Set 3 and Set 4.

Further embodiments will become apparent from the following examples which illustrate the invention in some of its major aspects but is not intended to limit the scope in any way thereof.

### EXAMPLES

### Example 1: Primer Design

Primer design is a crucial aspect of LAMP and RT-LAMP assays and is important for assay sensitivity and specificity, as well as the robustness and speed of the assay. Primer design is one of the biggest factors affecting the sensitivity and specificity of LAMP and RT-LAMP assays. The presence of 6 primers in one tube at very high concentrations means an increased risk of non-specific amplification which must be eliminated by careful primer design and screening of putative primer sets. Limited software options exist to assist in LAMP primer design, however these programs alone do not result in robust primer designs. In order to arrive at a suitable set of primers, particularly for a clinical assay, it is necessary to test many different primer sets empirically and identify the best performing primers. Here, sixteen different RT-LAMP primer sets were designed and tested against various target genes across the SARS-CoV-2 RNA genome, including open reading frames 1a, 1b, M, and N. The primer sets were systematically tested using synthetic SARS-CoV-2 whole genome RNA. The ability of the primer sets to amplify their respective target regions was assessed using the cycle threshold (“Ct”) value as an indicator of amplification speed. The Ct value represents the first cycle number at which a detectable signal for the amplified nucleic acid product is significantly above background. Typically this is a fluorescence signal. The primer sets were also evaluated to determine their limit of detection (LOD), an indicator of sensitivity and their specificity for the target nucleic acid by evaluating the amount of non-specific amplification using other respiratory viruses as template DNA in the reaction. We identified top performing primer sets as those able to detect at least 10 copies of synthetic SARS-CoV-2 RNA per microliter. Top performing primer sets were further optimized by re-designing one or more primers of the set to increase amplification speed and reduce cross-reactivity. The top performing primer sets are designated sets 1, 2, and 3 and are described in Table 1 above.

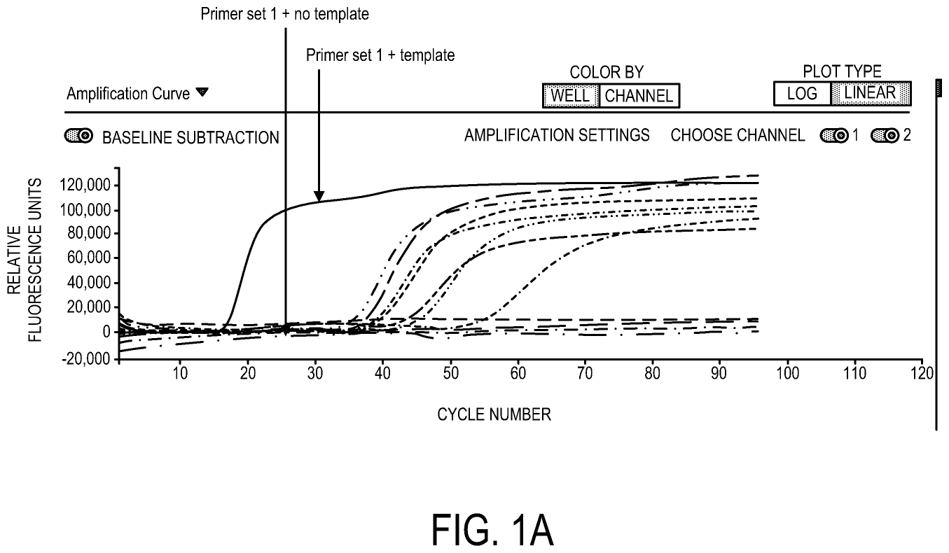

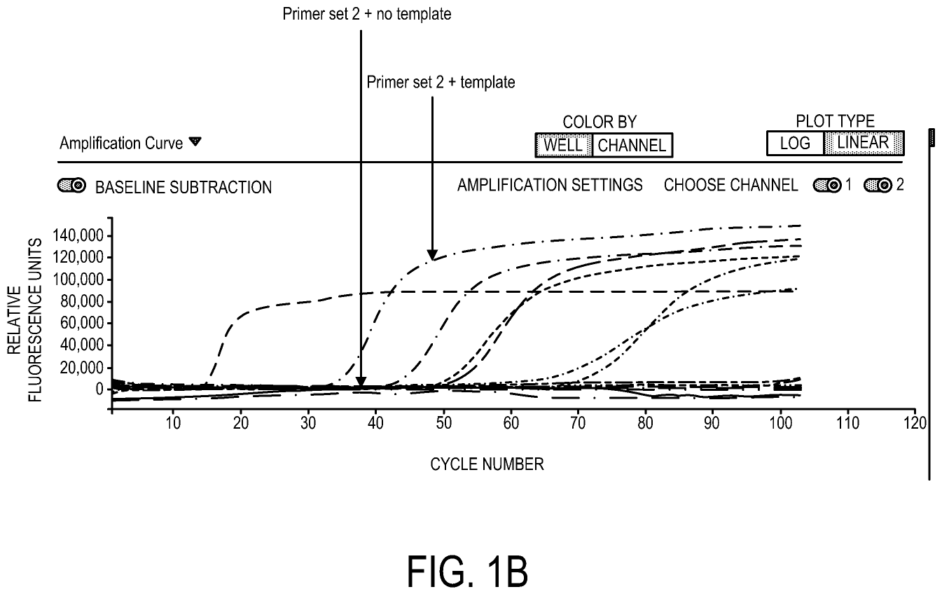

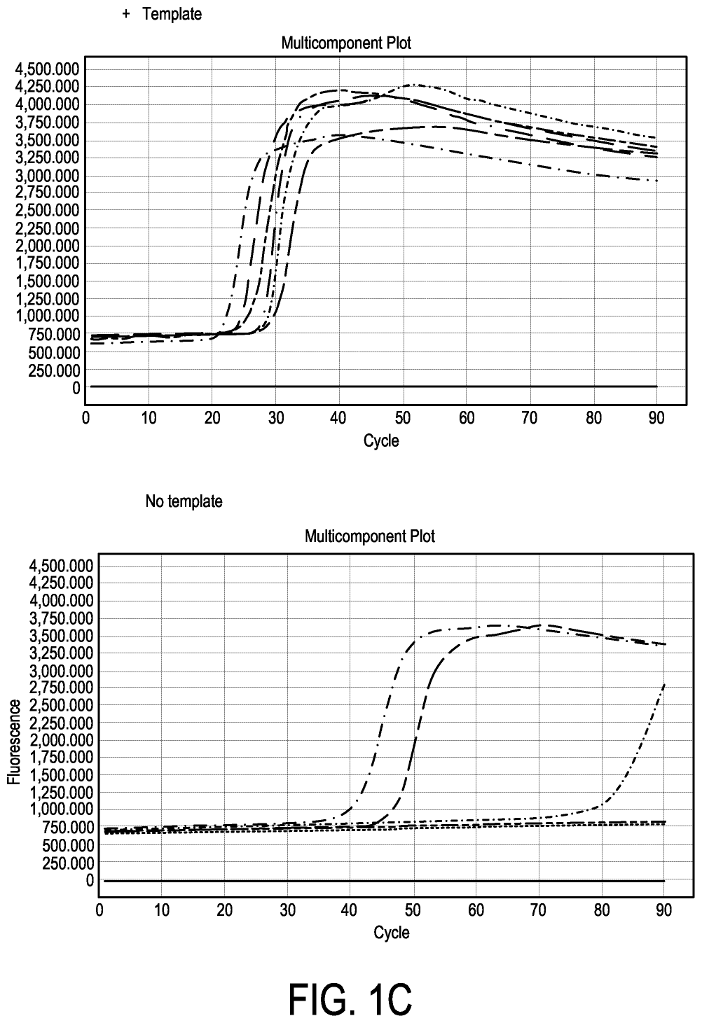

FIGS. 1A-B show representative amplification profiles for the 16 different primer sets empirically tested. In panels A and B, arrows indicate the top performing primer sets 1 and 2, respectively. FIG. 1C shows amplification profiles for primer set 3, which was designed by optimization of primer set 2. The reactions were performed at 65 C for 1 hour using synthetic SARS-CoV-2 whole genome RNA as the template at about 100 copies per microliter final concentration. A fluorescent DNA intercalating dye was used to detect amplified DNA.

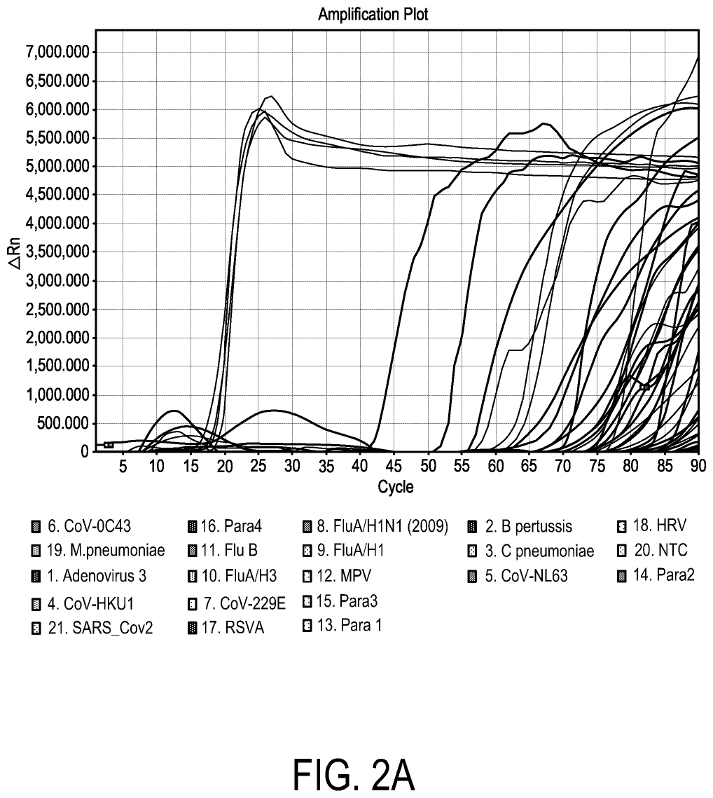

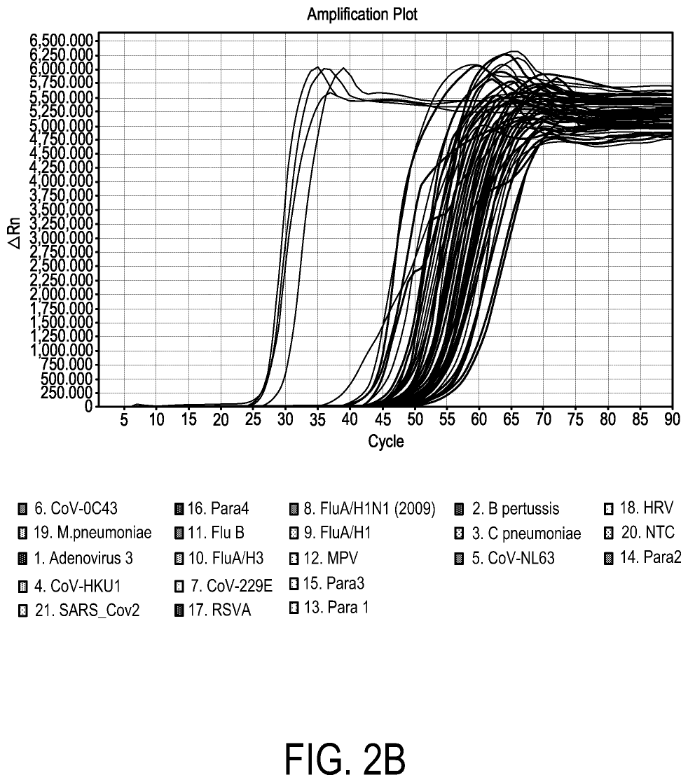

The specificity of the RT-LAMP primers for SARS-CoV-2 was characterized by testing against a panel of respiratory viruses. These included Influenza A H1N1 (A/NY/02/09), Parainfluenza Type 4A, Parainfluenza Type 4B, Rhinovirus (1A), Adenovirus Type 3, Influenza A H1 (A/New Caledonia/20/99), Respiratory Syncytial Virus A, Parainfluenza Type 1, Coronavirus NL63, Mycoplasma pneumoniae (M129), Influenza A H3 (A/Brisbane/10/07), Respiratory Syncytial Virus B (CH93(18)-18), Coronavirus 0C43, Coronavirus HKU-1, Influenza B, (B/Florida/02/06), Parainfluenza Type 3, Human Metapneumovirus (Peru6-2003), Legionella pneumophila (Philadelphia), Parainfluenza Type 2, Coronavirus 229E, Human Bocavirus Chlamydophila, pneumoniae (CWL-029). As shown in FIGS. 2A-B, primer sets 1 and 3 each demonstrated specific amplification towards SARS-CoV-2 with little cross-reactivity for other respiratory viruses.

### Example 2: Limit of Detection (LoD) of the Inactivated SARS-Cov-2 Viral Particles by RT-LAMP

We initially determined that the presence of RNases in saliva samples required the use of an RNase inhibitor for successful amplification of target RNA. We determined that the addition of an RNase inhibitor can completely preserve RNA in simulated saliva samples for up to one hour at room temperature. Simulated saliva consisted of normal saline containing saliva and RNase inhibitor spiked with different concentrations of inactivated SARS-Cov-2 virions. A direct assay using simulated saliva containing RNase inhibitor added directly to RT-LAMP reagents was able to detect either synthetic RNA or inactivated SARS-CoV-2 viral particles with an LOD 95% using 100 copies of RNA or viral particles per microliter. LOD 95% is defined as the number of copies of genomic RNA (or viral particles) per unit of volume (or per reaction) in which 95% of the tests are accurately detected by the assay. Freezing or heat-treatment of the samples was avoided. The RNA template was aliquoted into one-time use tubes (e.g., 1000 copies/μl) and kept in −80° C. until use. The RT-LAMP reagents comprised an appropriate buffer, e.g., saline, a set of oligonucleotide primers selected from primer set 1, 2, or 3 described above, deoxyribonucleotide triphosphates (dNTPs), a source of magnesium ions (e.g., MgCl₂ or MgSO₄), a DNA intercalating dye (e.g., Chai Green™, or Eva Green™), reverse transcriptase, and DNA polymerase.

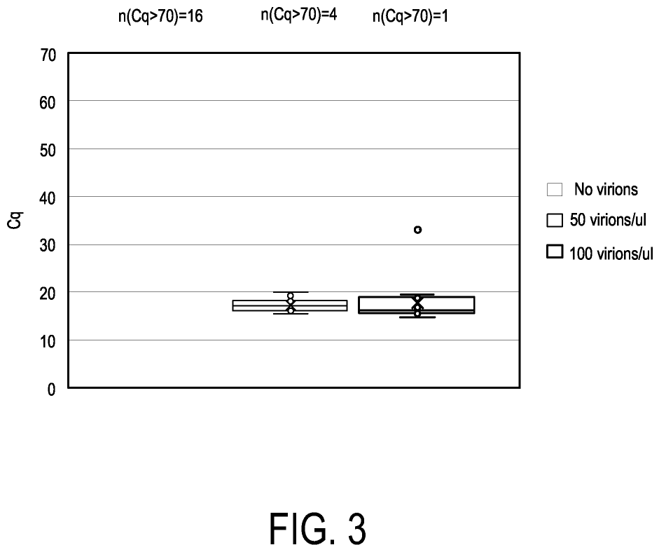

Each RT-LAMP reaction contained the RT-LAMP reagents and about 1% of a simulated saliva sample containing a predetermined amount of viral particles (either 50 or 100 virions per microliter). Reactions were incubated at 65° C. for one hour. Signal was monitored in the FITC channel to detect amplified product via incorporation of an intercalating DNA dye. Time to Cq for the positive sample was 15-25 minutes. Cq is the time needed to reach the half maximal signal. Signal in negative samples showed up after 60 minutes, but occasionally signals were generated after 30 minutes. Results are summarized in Table 2 below and in FIG. 3. These results show that the LoD 95% of this protocol is ˜400 copies/ul. The assay can still correctly detect 75% of the positive samples with viral load=50 copies/ul.

TABLE 2
Summary of direct assay (no lysis or heat
treatment of sample prior to RT-LAMP).

| | Virion conc. (virions/μl) | | |
|---|---|---|---|
| | 0 | 50 | 100 |
| Positive (Cq < 25) | 0/16 (0%) | 12/16 (75%) | 15/16 (94%) |
| Negative (Cq > 30) | 16/16 (100%) | 4/16 (25%) | 1/16 (6%) |
| Negative breakdown | | | |
| 30 < Cq < 45 | 0/16 | 0/4 | 1/1 |
| 45 < Cq < 60 | 0/16 | 0/4 | 0/1 |
| Cq > 60 | 16/16 | 4/4 | 0/1 |
| Median (Cq) | >70 | 17.2 | 16.24 |
| Mean (Cq) | >70 | 17.25 | 17.81 |
| Standard Deviation (Stdev) | NA | 1.41 | 4.31 |

### Saliva Sample Collection:

RNase inhibitor should be added to saliva samples as soon as possible after collection. Saliva samples can be collected in saline, water, or viral transfer media supplemented with RNase inhibitor. Prior to RT-LAMP reaction, samples may be stored at 4° C. and are stable at room temperature for up to 1 hour, although ideally the time between sample collection and RT-LAMP assay should be minimized.

### Example 3: LOD Improved with Addition of Lysis Step

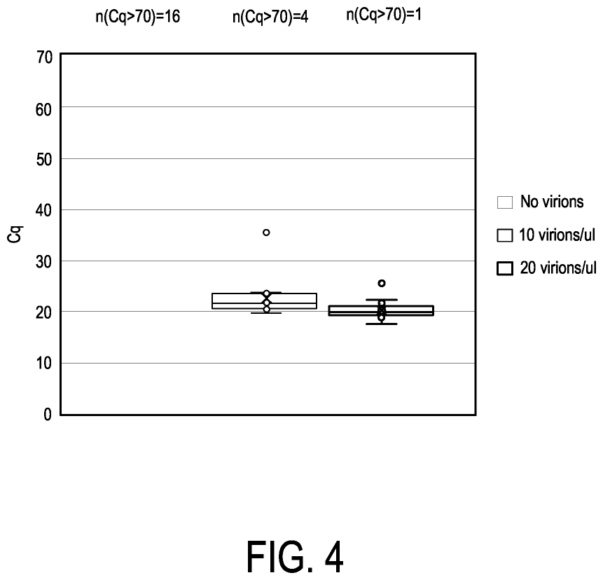

In further refining the assay to improve the LOD using inactivated SARS-CoV-2 viral particles, we determined that the LOD could be reduced by 5 to 10 fold (10-20 virions/μl) by addition of a separate lysis step. The optimal lysis was determined to occur with addition of a lysis buffer (e.g., QuickExtract™ DNA extraction buffer) containing RNase inhibitor (e.g., RiboGuard™) to the sample, followed by heat treatment at 95° C. for 3 minutes. Lysed sample was then added directly to the RT-LAMP reaction which contained the same reagents as described in the previous examples. Results are summarized in Table 3 below and in FIG. 4. These results show that the LoD 95% of with this modified protocol can be improved to ˜20 copies/ul. The protocol can still correctly detect 75% of the positive samples with viral load=10 copies/ul.

TABLE 3
Summary of assay using lysis and heat
treatment of sample prior to RT-LAMP.

| | Virion conc. (virions/μl) | | |
|---|---|---|---|
| | 0 | 10 | 20 |
| Positive (Cq < 25) | 0/16 (0%) | 12/16 (75%) | 15/16 (94%) |
| Negative (Cq > 30) | 16/16 (100%) | 4/16 (62.5%) | 1/16 (6%) |
| Negative breakdown | | | |
| 30 < Cq < 45 | 0/16 | 1/4 | 0/1 |
| 45 < Cq < 60 | 0/16 | 0/4 | 0/1 |
| Cq > 60 | 16/16 | 3/4 | 1/1 |
| Median (Cq) | >70 | 21.55 | 19.44 |
| Mean (Cq) | >70 | 22.72 | 20.04 |

### Example 4: LOD Further Improved with Addition of Centrifugal Filtration Step

Isothermal DNA/RNA amplification methods such as LAMP, RT-LAMP, RPA, etc. have been previously used for the detection of various pathogens in different types of samples (blood, saliva, tissue, environmental samples, etc.) in both laboratory and POC settings (Shi, Li et al. 2011, Panek and Frąc 2019, Silva, Paiva et al. 2019). However, in order to achieve good sensitivity by these methods, similar to PCR-based approaches, a nucleic acid (RNA/DNA) purification step is needed (i.e., silica bead or column-based purification, phenol-chloroform extraction, ethanol precipitation, etc.). Since the purification step is often needed to be performed in laboratory settings with trained personnel, POC assays often sacrifice sensitivity over ease of operation.

To further increase the sensitivity of the present RT-LAMP assay and make it on par with RT-qPCR assays (the gold standard of molecular diagnostics assays), the starting material needed to be further purified. Most sensitive laboratory assays use RNA purification as a critical step in their workflow to achieve high sensitivity. RNA purification allows the removal of cellular impurities and inhibitors that may degrade the RNA and/or inhibit the enzymatic reactions of the assay, e.g., the reverse transcriptase and DNA polymerase reactions in RT-LAMP and RT-qPCR assays. However, state-of-the-art RNA purification protocols using silica beads and columns or phenol-chloroform extraction require extensive hands-on time, expertise, and costly reagents. Those workflows need to be performed by trained personnel in laboratory settings and are challenging to scale or to be used in POC settings.

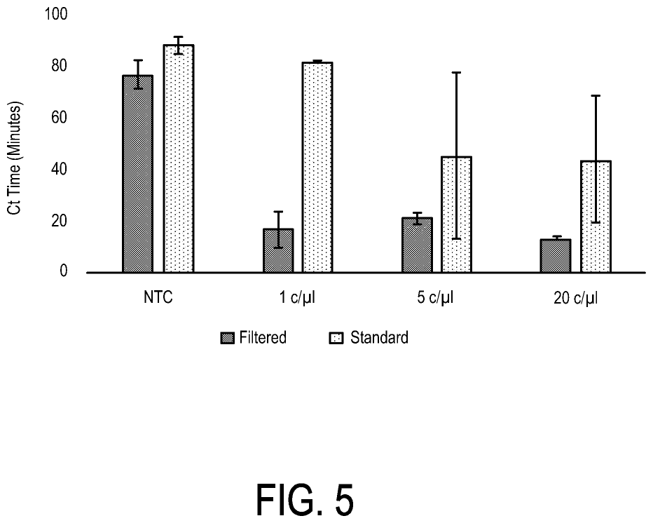

To address these challenges and increase the sensitivity of our assay, we substituted the standard RNA extraction/purification steps with a new step designed to capture the viral particles by leveraging the differences in the physical properties of the virions compared to the impurities in the starting biological sample. Various strategies were tested before arriving at a method using simultaneous ultrafiltration by air pressure (using e.g., 200 MWCO filters) and centrifugal force to effectively purify and concentrate the viral particles. Surprisingly, performing this step of simultaneous ultrafiltration and centrifugation prior to subjecting the sample to chemical lysis and heat treatment at 95° C. markedly improved the sensitivity of the assay, without the need for a standard RNA extraction step. This optimized assay performed with an LOD (95%) of 1-5 copies/μl, which marks a reduction of about 4-10 fold compared with the assay in Example 3. FIG. 5 illustrates these results and shows that centrifugal filtration enables sensitive detection of SARS-CoV-2 with RT-LAMP.

### Example 5: Illustrative Clinical Protocol

In an exemplary embodiment of a clinical protocol using the assay described in Example 4, twenty-seven 8-tube PCR strips compatible with a desktop qPCR machine are filled with RT-LAMP reagents, as described above. Each PCR strip is intended for a single patient sample. Two different RT-LAMP reactions are used, each containing a different primer set. This is intended to provide robust results of high confidence for clinical use. Thus, the first four tubes in each strip contain two positive and two negative controls, one each for each RT-LAMP reaction. The remaining four tubes may contain duplicates of each of the two RT-LAMP reactions reserved for patient sample. In practice, the PCR strips may be provided with the tubes pre-loaded with the required RT-LAMP reagents and controls, so that the only steps needing to be performed are the patient sample collection and pre-processing steps followed by aliquoting patient sample into each of the patient sample tubes.

TABLE 4
Representative PCR strip utilization

| Tube ID | 1 | 2 | 3 | 4 | 5 | 6 | 7 | 8 |
|---|---|---|---|---|---|---|---|---|
| Condition | Positive | Positive | Negative | Negative | Sample | Sample | Sample | Sample |
| | | | | | | | replicate | replicate |
| | | | | | | | (optional) | (optional) |
| RT-LAMP | primer | primer | primer | primer | primer | primer | primer | primer |
| Reaction | set 1 | set 3 | set 1 | set 3 | set 1 | set 3 | set 1 | set 3 |
| Volume | 20 μl | 20 μl | 20 μl | 20 μl | 15 μl | 15 μl | 15 μl | 15 μl |

Sample Collection and Pre-Processing

Samples are collected in saline by dipping the collection swab into a first collection tube containing 1 ml saline solution and RNase inhibitor. An aliquot, e.g., 500 μL, of the saline sample is then added onto a filter assembly contained in a second collection tube, e.g., an Amicon or Vivaspin centrifugal filter assembly, 100 kDa MWCO. The second collection tube containing the sample and filter assembly is subjected to centrifugation, e.g., at 14000×g or 15000×g for 20 minutes. Following centrifugation, about 10-15 μL of sample concentrate is retained in the filter assembly and the remaining flow-through is discarded. Sample concentrate is aliquoted into prepared PCR tubes containing lysis buffer, e.g., 4 μl of the concentrate into 16 μl lysis buffer. The lysis buffer may be a commercially available buffer or a standard cell lysis buffer, e.g., containing surfactant such as 1% Triton-X and 4M guanidine hydrochloride in a suitable Tris buffer such as 50 mM Tris HCl pH 7.4. Other similar Triton™ detergents may also be used, or a Tween™ detergent such as Tween 20 or Tween 80, or a maltoside such as n-dodecyl-β-D-maltoside. Likewise, other suitable buffering agents, in addition to Tris, may include CAPS, HEPES, MOPS, PIPES, TAPS, TE, TES, or Tricine. Optionally, additional RNase inhibitor may be added to the lysis buffer. The sample is then subjected to heat treatment at 95° C. for 3 minutes, e.g., using a thermocycler or a heat block. Immediately following heat treatment, an aliquot, e.g., 5 μL, of the sample is added to RT-LAMP reagents or stored at 4° C. RT-LAMP is performed at 65° C. for 90 cycles (one minute per cycle).

Using primer sets 1, 2, or 3, under laboratory settings, positives amplify with a Cq=10-30 minutes, and negatives amplified at later times, either more than 30 minutes or more than 50 minutes. Patient samples, which may contain additional substances that inhibit the RT-LAMP reaction, may also require additional time. For example, patient samples may require about 30-50 minutes for a positive test, while a negative test would produce a signal in more than 90 minutes.

### Example 6: Illustrative Protocol and Kit for Nasal Swab Sample

In embodiments, the disclosure provides a kit suitable for collection of a sample from a nasal swab. In an embodiment, the kit contains a tube assembly comprising a reaction tube and lyophilized reagents, which comprise reagents necessary for multiplex detection of SARS-CoV-2 RNA and an internal control human RNA, such as Beta-actin (ActB) RNA. The internal control confirms the presence of human cellular material in the collected nasal specimen and further controls for proper assay execution, independent of the presence or absence of target SARS-CoV-2 RNA in the sample.

In embodiments, the lyophilized reagents comprise primers for reverse transcription and DNA amplification of the membrane protein (M) region of the SARS-CoV-2 RNA and an internal control human RNA, such as ActB (beta actin) RNA, and optionally one or more of a buffering agent, a non-ionic surfactant, and salts such as KCl and ammonium sulfate. In embodiments, the lyophilized reagents further comprise a reverse transcriptase, DNA polymerase, and/or RNase inhibitors. In embodiments, the primers comprise a set of 4-6 primers directed to the M region of SARS-CoV-2 RNA and at least one detection primer, such as a DARQ primer. In embodiments, the primers comprise primer set 4 described in Table 1. In embodiments, the lyophilized reagents further comprise a fluorogenic DNA intercalating dye, such as a SYBR green or Chai Green™, or similar dye such as SYBR Green I and II, SYBR Safe, SYBR Gold, Eva Green, Ethidium Bromide, Oxazole yellow-based cyanine dyes (e.g. YOYO-1, DiYO-1, TOTO-1, DiTO-1, TOTO-3), Pico Green, SYTO 9, SYTO 13, SYTO 16, SYTO 60, SYTO 62, SYTO 64, SYTO 82, Boxto, Miami Green, Miami Yellow, or Miami Orange, to produce a fluorescence signal upon successful reverse transcription and DNA amplification of the target region of the SARS-CoV-2 RNA, such as the membrane protein (M), and/or the internal control RNA, such as human ActB (beta actin) RNA. In accordance with embodiments comprising both a DARQ primer and a fluorogenic DNA intercalating dye, the DARQ and DNA intercalating dye fluorescence wavelengths are different such that simultaneous detection of both signals is possible in the reaction. The fluorogenic DNA intercalating dye serves as a measure to ensure that the integrity of the reagents in the assay is not compromised. In other embodiments, the lyophilized reagent mix further comprises buffering agents, salts (e.g., KCl and (NH₄)₂SO₄), and a non-ionic surfactant (e.g., Tween 20). In embodiments, the lyophilized reagent mix further comprises one or more of an RNase inhibitor, a serum albumin (e.g, bovine serum albumin “BSA”), a reducing agent such as tris(2-carboxyethyl)phosphine (TCEP) and salts thereof, e.g., TCEP-HCl, and thermolabile uracil-DNA glycosylase (UDG).

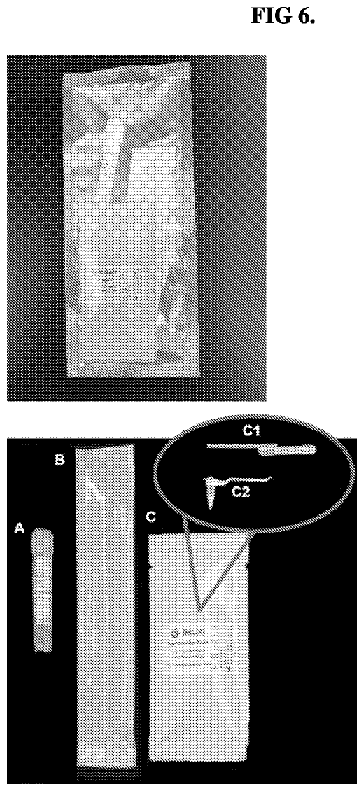

The kit may also optionally further contain one or more of a collection swab, a collection tube containing a buffer for sample elution and lysis, and a transfer pipette. In an embodiment, the buffer comprises a detergent, a buffer, and an RNase inhibitor, such as PVSA, and nuclease-free water. An exemplary kit is illustrated in FIG. 6.

The following illustrates an exemplary workflow for sample collection and processing.

Anterior nasal (AN) swab sample collection: a sterile swab is inserted into one nostril until a resistance is met and is rubbed against the inside wall of the nostril for ˜10 seconds. The same process is repeated for the other nostril (this can be either self-collected or healthcare worker-administered)

Sample elution: the AN swab sample is eluted in an elution buffer by swirling the swab in the buffer, for example by swirling 30 times.

Sample transfer: a single-use transfer pipette is used to move from 75-200 uL, for example about 100 uL, of the eluted sample to a tube assembly containing a lyophilized mixture of reagents to perform RT-LAMP and a stainless steel ball bearing which is used to mix the solution inside the test tube during the RT-LAMP reaction.

RT-LAMP+signal readout: the test tube is inserted into a suitable instrument for reaction at 67° C. for 25-30 minutes. During the reaction, fluorescence signal in the FAM and HEX channels is measured once every 20 seconds.

### Example 7: Results for Primer Set 4

The following example demonstrates the successful amplification and detection of two different nucleic acid targets in a single reaction mixture, with the membrane protein (M) region of the SARS-CoV-2 RNA as the first target and human beta actin (ActB) RNA as the second target. The technique utilized both a DNA intercalating dye, Chai Green™ in this case, and detection primers adapted to contain a quencher-fluorophore duplex region that emits a fluorescence signal upon strand separation, for the detection of the amplified targets of the primers, referred to as DARQ primers. An increase in fluorescence signal of the DNA intercalating dye indicates that either or both of the two different nucleic acid targets are amplified, while the DARQ primers are designed to produce a signal only when one of the two nucleic acid targets, such as the membrane protein (M) region of the SARS-CoV-2 RNA, is amplified. In these experiments, the DARQ primers and the DNA intercalating dye emit their fluorescence signals at different wavelengths, enabling the two signals to be detected separately.

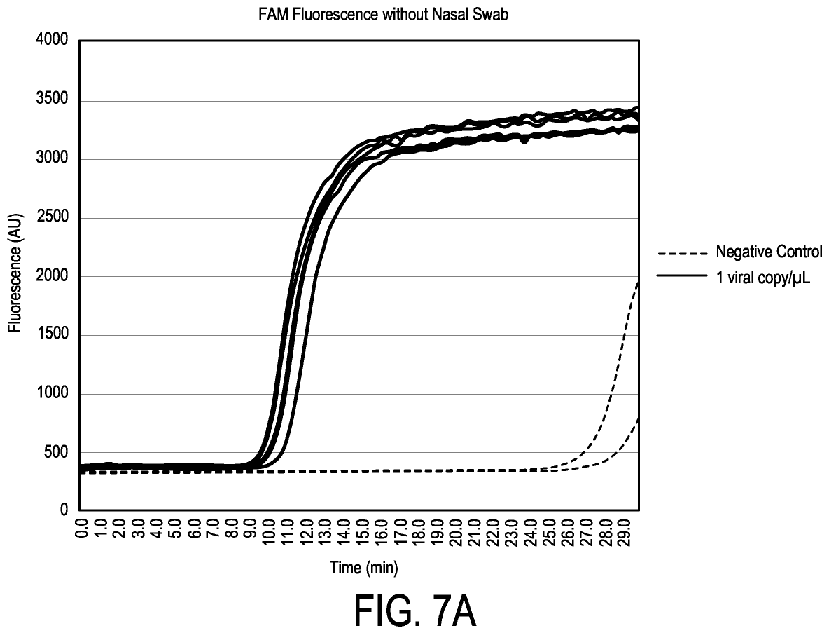

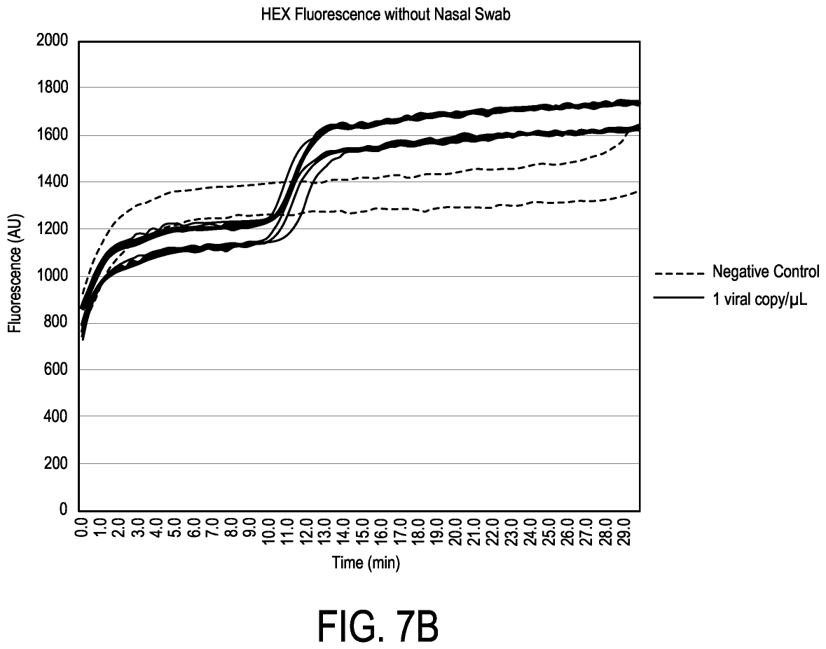

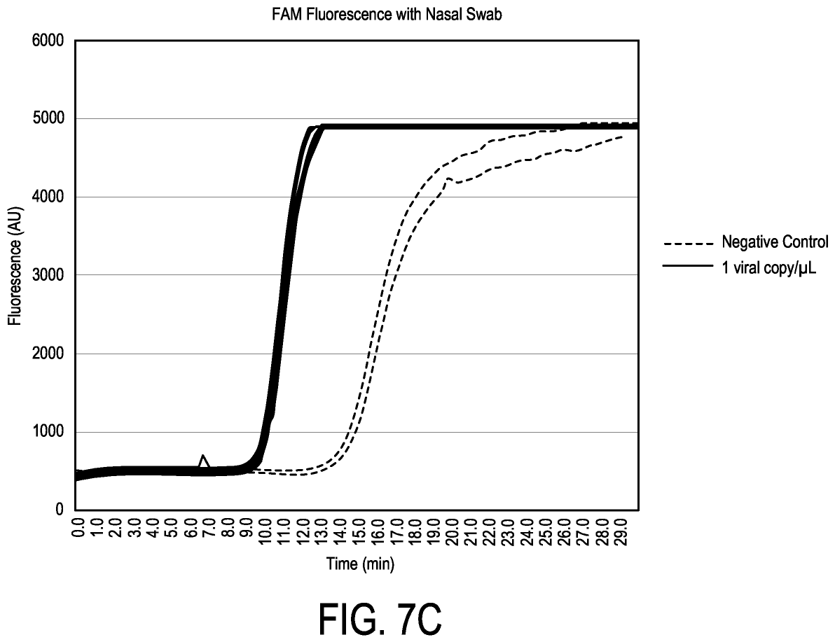

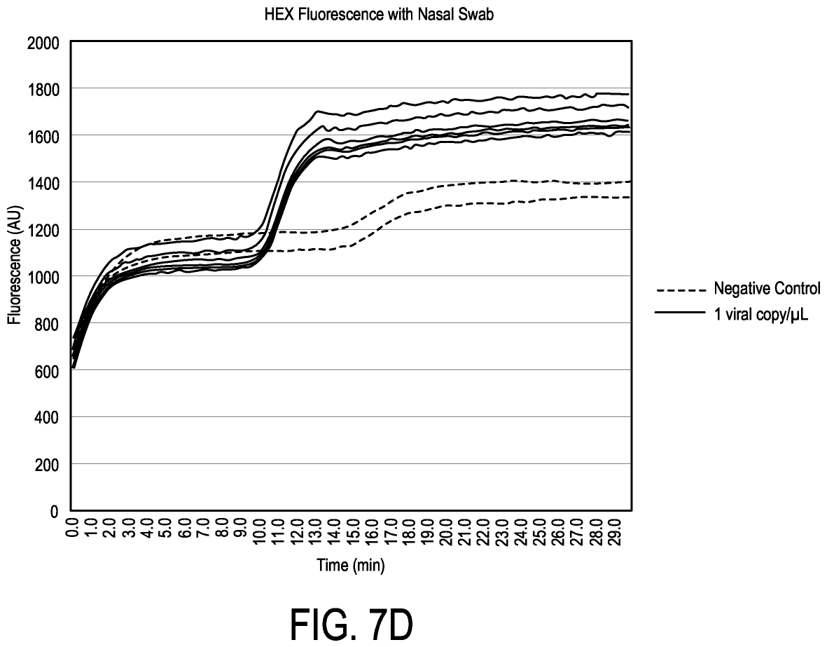

Amplification curves of negative and positive samples with or without nasal swab sample are shown in FIG. 7. Without a nasal swab sample, fast FAM channel amplification (T_{d}<26 min.), as seen in the 1 viral copy/μL tests, represents the presence of SARS-CoV-2 virus and late FAM channel amplification (T_{d}>26 min.) signifies false amplification and is disregarded (FIG. 7A). Without a nasal swab sample, fast HEX channel amplification (T_{d}<26 min.), as seen in the 1 viral copy/μl, tests, represents the presence of SARS-CoV-2 virus while late HEX channel amplification (T_{d}>26 min.) signifies false amplification and is disregarded (FIG. 7B). Without a nasal swab sample, absence of both FAM and HEX channel signals within 26 minutes indicates the absence of virus in the sample. With a nasal swab sample, fast FAM channel amplification (T_{d}<26 min.) represents the general presence of beta actin and/or SARS-CoV-2 virus (FIG. 7C). With a nasal swab sample, fast HEX channel amplification (T_{d}<26 min.), as seen in the 1 viral copy/μL tests, represents the presence of SARS-CoV-2 virus and fast FAM channel amplification (T_{d}<26 min.) with late or no HEX channel amplification signifies a nasal swab sample with no detectable SARS-CoV-2 virus (FIG. 7D).

In summary, where HEX detects amplified virus and FAM detects amplified DNA that could be either viral or the internal control, beta-actin (ActB), the following describes the interpretation of assay results:
HEX+ FAM+: indicates presence of the virus;
HEX− FAM+: indicates the virus is not present;
HEX− FAM−: inconclusive, the assay should be re-run; and
HEX+ FAM− inconclusive, the assay should be re-run.

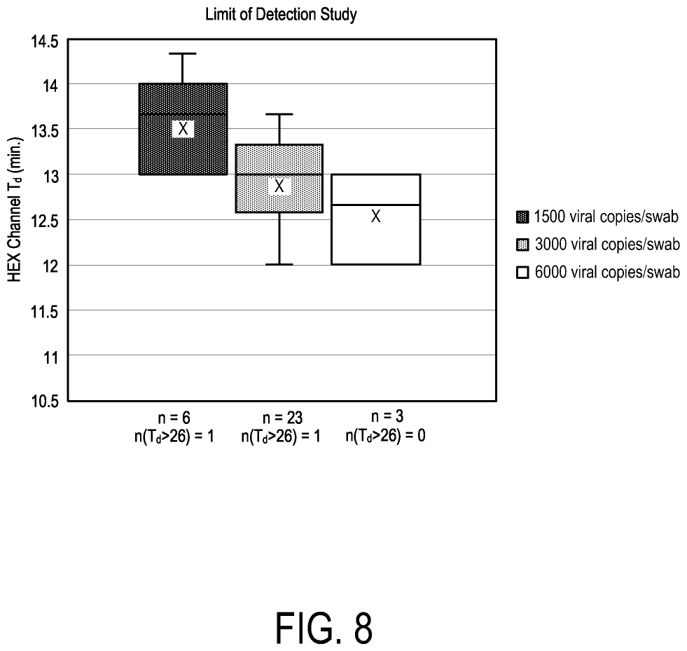

The limit of detection (LOD) study shown in FIG. 8 indicates that the LOD of the assay is 3000 viral copies/swab. Range finding experiments were performed for 1500, 3000, and 6000 viral copies/swab. There was 1 sample with 1500 copies/swab and 1 sample with 3000 copies/swab that had a HEX channel T_{d}>26 min., which is called “negative” by our test algorithm. 3000 copies/swab was determined to be the LOD after 22 out of 23 replicates were correctly called “positive” by our test algorithm (>95%, threshold set by the U.S. Food and Drug Administration).

Cross Reactivity. The test was run with potentially cross-reactive organisms. Each organism was prepared at the concentration listed in Table 5 in a negative clinical nasal matrix, and 50 μL was pipetted onto a fresh, unused nasal swab. Each organism was tested in triplicate. No organisms were found to be cross-reactive.

TABLE 5
Summary of cross-reactivity with other organisms.

| | | # Pos/ | Cross |
|---|---|---|---|
| Organism Tested | Concentration Tested | # Tested | Reactive |
| Adenovirus 1 | 3.09 × 10\hat{ }7 TCID50/ml | 0/3 | No |
| Human Metapneumovirus (hMPV) | 3.8 × 10\hat{ }5 TCID50/mL | 0/3 | No |
| Parainfluenza virus 1 | 1.26 × 10\hat{ }5 TCID50/ml | 0/3 | No |
| Parainfluenza virus 2 | 1.51 × 10\hat{ }5 TCID50/ml | 0/3 | No |
| Parainfluenza virus 3 | 3.39 × 10\hat{ }6 TCID50/ml | 0/3 | No |
| Parainfluenza virus 4a | 1.41 × 10\hat{ }4 TCID50/ml | 0/3 | No |
| Influenza A | 4.17 × 10\hat{ }4 TCID50/ml | 0/3 | No |
| Influenza B | 4.68 × 10\hat{ }3 TCID50/ml | 0/3 | No |
| Enterovirus 68 (e.g., EV68) | 1.51 × 10\hat{ }5 TCID50/ml | 0/3 | No |
| Enterovirus 71 | 4.17 × 10\hat{ }4 TCID50/ml | 0/3 | No |
| RSV A | 1.05 × 10\hat{ }5 TCID50/mL | 0/3 | No |
| RSV B | 1.55 × 10\hat{ }3 TCID50/mL | 0/3 | No |
| Rhinovirus type 1A | 1.41 × 10\hat{ }4 TCID50/mL | 0/3 | No |
| Pneumocystis jirovecii (PJP) | 6.34 × 10\hat{ }7 CFU/mL | 0/3 | No |
| Human coronavirus OC43 | 5.01 × 10\hat{ }4 TCID50/mL | 0/3 | No |
| Human coronavirus NL63 | 1.70 × 10\hat{ }4 TCID50/mL | 0/3 | No |
| MERS-coronavirus | 4.17 × 10\hat{ }4 TCID50/mL | 0/3 | No |
| SARS-coronavirus | 10 fold dilution of 27.62 Ct | 0/3 | No |
| Candida albicans | 2.09 × 10\hat{ }7 cfu/ml | 0/3 | No |
| Pseudomonas aeruginosa | 6.64 × 10\hat{ }6 cfu/ml | 0/3 | No |
| Staphylococcus epidermidis | 9.0 × 10\hat{ }7 cfu/ml | 0/3 | No |
| Streptococcus salivarius | 1.2 × 10\hat{ }7 cfu/mL | 0/3 | No |
| Human coronavirus 229E | 1.26 × 10\hat{ }5 TCID50/mL | 0/3 | No |
| Haemophilus influenzae | 5.1 × 10\hat{ }7 cfu/mL | 0/3 | No |
| Legionella pneumophila | 2.4 × 10\hat{ }7 cfu/mL | 0/3 | No |
| Streptococcus pyogenes | 1.2 × 10\hat{ }8 cfu/mL | 0/3 | No |
| Bordetella pertussis | 1.95 × 10\hat{ }8 cfu/mL | 0/3 | No |
| Mycoplasma pneumoniae | 4.4 × 10\hat{ }6 cfu/mL | 0/3 | No |
| Mycobacterium tuberculosis | 4.3 × 10\hat{ }4 genome copies/μL | 0/3 | No |
| Streptococcus pneumoniae | 4.1 × 10\hat{ }4 genome copies/μL | 0/3 | No |
| Chlamydophila pneumoniae | 2.11 × 10\hat{ }7 IFU/mL | 0/3 | No |
| Human coronavirus HKU1 | 5.5 × 10\hat{ }4 genome copies/μL | 0/3 | No |

Interfering Substances. The test was run with three positive and three negative replicates for each interfering substance. Each substance was prepared at the concentration listed in Table 6 in a negative clinical nasal matrix, and 50 μL was pipetted onto a fresh, unused nasal swab. To prepare a positive replicate, inactivated SARS-CoV-2 viral particles were spiked into the interfering substance-containing negative clinical matrix, and 50 μL of the spiked clinical matrix was pipetted onto a fresh, unused nasal swab. If a test returned an invalid result, it was repeated with a new test kit. If the test continued to return an invalid result, the interfering substance was diluted and the testing process was repeated. None of the substances produced an anomalous result at the concentrations tested.

TABLE 6
Summary of performance with potentially interfering substances.

| | | | # Neg/ | # Pos/ |
|---|---|---|---|---|
| | Concentration | | # Neg | # Pos |
| Substance | Tested | | expected | expected |
| Afrin Nasal Spray | 15% | v/v | 3/3 | 3/3 |
| Whole blood | 1% | v/v | 3/3 | 3/3 |
| Chloraseptic Max | 5% | v/v | 3/3 | 3/3 |
| Flonase Allergy Relief | 1% | v/v | 3/3^{†} | 3/3 |
| Mupirocin | 5 | mg/mL | 3/3* | 3/3 |
| NeoSynephrine Cold and Sinus | 2.5% | v/v | 3/3* | 3/3 |
| Extra Strength Spray | | | | |
| Zanamivir | 250 | μg/mL | 3/3* | 3/3 |
| Ayr Saline Nasal Mist | 2.5% | v/v | 3/3* | 3/3 |
| Zicam Extreme Congestion | 2.5% | v/v | 3/3 | 3/3 |
| Relief | | | | |
| Oseltamivir Phosphate | 10 | μg/mL | 3/3 | 3/3 |
| Tobramycin | 5 | μg/mL | 3/3* | 3/3 |
| Robitussin | 5% | v/v | 3/3 | 3/3 |
| Cepacol Lozenges | 3 | mg/mL | 3/3 | 3/3 |

*One replicate repeated with a new DxLab COVID-19 test kit due to an initial invalid result
^{†}Displayed interference at the initial testing concentration of 2.5% v/v. Additional dilutions were tested, and 1% v/v was determined to be the concentration at which there was no interference.

### EQUIVALENTS

Those skilled in the art will recognize or be able to ascertain using no more than routine experimentation, many equivalents to the specific embodiments of the invention described herein. Such equivalents are intended to be encompassed by the following claims.

All references cited herein are incorporated herein by reference in their entirety and for all purposes to the same extent as if each individual publication or patent or patent application was specifically and individually indicated to be incorporated by reference in its entirety for all purposes.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

What is claimed is:

1. A method for detecting an organism in a biological sample of a human subject, the method comprising
subjecting the biological sample to chemical lysis by contacting the sample, or an apparatus comprising the sample, with a lysis buffer comprising a buffering agent, an RNase inhibitor, and a non-ionic surfactant to produce a lysed sample;
subjecting the lysed sample to an isothermal nucleic acid amplification reaction performed in a reaction mixture comprising
a first set of 4-6 oligonucleotide primers directed to a first target nucleic acid which is a nucleic acid of the organism, a second set of 4-6 oligonucleotide primers directed to a second target nucleic acid which is a nucleic acid of the human subject,
at least two different reagents suitable for independent detection of amplified target nucleic acids,
a mixture of deoxyribonucleotide triphosphates (dNTPs), a source of magnesium ions, an optional reverse transcriptase, and a DNA polymerase or a dual specificity reverse transcriptase/DNA polymerase,
and detecting amplified target nucleic acids in the reaction mixture by detecting a signal from each of the at least two different reagents for detection of amplified target nucleic acids,
wherein detection of at least two different signals indicates presence of the organism in the sample.

2. The method of claim 1, wherein the isothermal nucleic acid amplification is a loop-mediated isothermal nucleic acid amplification reaction (LAMP) or RT-LAMP reaction.

3. The method of claim 2, wherein the at least two different reagents suitable for independent detection of amplified target nucleic acids comprises at least one set of detection primers adapted to contain a quencher-fluorophore duplex region, wherein the set of detection primers is directed to either the first or second target nucleic acid.

4. The method of any one of claims 1-3, wherein the second target nucleic acid is a polynucleotide of a human gene that is constitutively expressed in human cells, optionally an RNase P or a beta-actin DNA or RNA.

5. The method of any one of claims 1-4, wherein the one or more reagents for detection of the amplified target nucleic acid comprises a fluorescent DNA intercalating dye, optionally selected from the group consisting of Chai Green™, SYBR Green I and II, SYBR Safe, SYBR Gold, Eva Green, Ethidium Bromide, Oxazole yellow-based cyanine dyes (e.g. YOYO-1, DiYO-1, TOTO-1, DiTO-1, TOTO-3), Pico Green, SYTO 9, SYTO 13, SYTO 16, SYTO 60, SYTO 62, SYTO 64, SYTO 82, Boxto, Miami Green, Miami Yellow, and Miami Orange.

6. The method of any one of claims 1-5, wherein the reaction mixture further comprises one or more of an RNase inhibitor, a serum albumin, a reducing agent such as tris(2-carboxyethyl)phosphine (TCEP) and salts thereof, and a uracil-DNA glycosylase (UDG).

7. The method of any one of claims 1-6, wherein the reaction mixture is lyophilized and the method further comprises a step of reconstituting the reaction mixture in a volume of an aqueous solution.

8. The method of claim 7, wherein the lyophilized reaction mixture further comprises one or more of dextran, mannitol, sorbitol, maltodextrin, trehalose, lactose, and/or lactitol.

9. The method of any one of claims 1-8, wherein the isothermal nucleic acid amplification reaction takes place at a constant temperature of 63° C., 64° C., 65° C., 66° C., 67° C., 68° C., 69° C., 70° C., 71° C., or 72° C.

10. The method of any one of claims 1-9, wherein the method is performed within a period of time from 10 to 45 minutes.

11. The method of any one of claims 1-10, wherein the method further comprises one or more additional steps prior to performing the isothermal nucleic acid amplification reaction, the one or more additional steps comprising one or more of a clarification step comprising simultaneous ultrafiltration and concentration to produce a clarified sample, and/or thermal treatment.

12. The method of claim 11, wherein the clarification step comprises centrifugation at a speed of from about 10,000 to 100,000×g, or from 10,000 to 20,000×g, or from 14,000 to 15,000×g, for from 5 to 20 minutes.

13. The method of claim 12, wherein the clarification step comprises ultrafiltration through a filter comprising a membrane having a molecular weight cut-off (MWCO) of from about 1 kDa to 300 kDa, preferably 3 kDa to 200 kDa, most preferably 100 kDa to 200 kDa.

14. The method of any one of claims 11-13, wherein the RNase inhibitor is polyvinylsulfonic acid (PVSA).

15. The method of claim 14, wherein the non-ionic surfactant is selected from polyethylene glycol p-(1,1,3,3-tetramethylbutyl)-phenyl ether, polyoxyethylene (20) sorbitan monolaurate, and a maltoside such as n-dodecyl-β-D-maltoside.

16. The method of any one of claims 11-15, wherein the thermal treatment comprises heating the sample to about 95° C. for a period of time from 30 seconds to 10 minutes, preferably 30 seconds to 5 minutes, most preferably 1 minute to 3 minutes.

17. The method of any one of claims 1-16, wherein the target nucleic acid comprises one or more RNA or DNA molecules of an organism selected from a virus, a bacterium, a fungus or a parasite.

18. The method of claim 17, wherein the target nucleic acid is of a virus.

19. The method of claim 18, wherein the virus is selected from severe acute respiratory syndrome coronavirus (SARS-CoV), Middle East respiratory syndrome coronavirus (MERS-CoV), and severe acute respiratory syndrome coronavirus 2 (SARS-CoV-2).

20. The method of claim 19, wherein the virus is SARS-Cov-2.

21. The method of claim 20, wherein the first set of oligonucleotide primers is selected from the group consisting of Set 1, Set 2, Set 3, and Set 4, identified by SEQ ID NOs 1-6 (Set 1), SEQ ID NOs 7-12 (Set 2), SEQ ID NOs 7, 9, 11, 13-15 (Set 3), and SEQ ID NOs 7-12, 16, 17 (Set 4); or SEQ ID NOs 1-4 of Set 1, SEQ ID NOs 7-10 of Set 2, SEQ ID NOs 7, 13, 9, and 14, of Set 3, or SEQ ID NOs 7-10 and SEQ ID NOs 16 and 17 of Set 4.

22. The method of any one of claims 19-21, wherein the biological sample is an upper respiratory tract sample, optionally wherein the upper respiratory tract sample is obtained from a nasopharyngeal (NP) swab, an oropharyngeal (OP) swab, a nasal swab, a nasal aspirate, or a nasal wash, further optionally wherein the nasal swab is an anterior nasal swab or a mid-turbinate nasal swab.

23. A method for detecting a SARS-CoV-2 virus in an upper respiratory tract sample obtained from a human subject, the method comprising subjecting the sample to a reverse transcription and loop-mediated isothermal nucleic acid amplification reaction (RT-LAMP) to amplify a first target nucleic acid of the SARS-CoV-2 virus utilizing a first set of oligonucleotide primers selected from the group consisting of Set 1, Set 2, Set 3, and Set 4, identified by SEQ ID NOs 1-6 (Set 1), SEQ ID NOs 7-12 (Set 2), SEQ ID NOs 7, 9, 11, 13-15 (Set 3), and SEQ ID NOs 7-12, 16, 17 (Set 4), and detecting the amplified first target nucleic acid, wherein detection of the amplified first target nucleic acid indicates presence of the SARS-CoV-2 virus in the biological sample.

24. The method of claim 23, wherein the RT-LAMP reaction is performed in a reaction mixture comprising the first set of oligonucleotide primers and a second set of oligonucleotide primers directed to a second target nucleic acid which is a nucleic acid of the human subject, optionally wherein the second target nucleic acid is a polynucleotide of a beta-actin RNA molecule.

25. The method of claim 24, wherein the reaction mixture further comprises a set of detection primers adapted to contain a quencher-fluorophore duplex region, wherein the set of detection primers is directed to either the first or second target nucleic acid.

26. The method of claim 25, wherein the reaction mixture further comprises a fluorescent DNA intercalating dye that emits a fluorescent signal at a different wavelength than the fluorophore in the detection primer.

27. The method of claim 26, wherein the reaction mixture further comprises a mixture of deoxyribonucleotide triphosphates (dNTPs), a source of magnesium ions, a reverse transcriptase, a DNA polymerase or a dual specificity reverse transcriptase/DNA polymerase, and one or more of an RNase inhibitor, a serum albumin, a reducing agent such as tris(2-carboxyethyl)phosphine (TCEP) and salts thereof, and thermolabile uracil-DNA glycosylase (UDG).

28. The method of claim 27, wherein the reaction mixture is lyophilized and the method further comprises a step of reconstituting the reaction mixture in a volume of an aqueous solution, optionally wherein the lyophilized reaction mixture comprises one or more of dextran, mannitol, sorbitol, maltodextrin, trehalose, lactose, and/or lactitol.

29. A kit for detection of a SARS-CoV-2 virus, the kit comprising
a first container comprising a first set of reagents for sample elution and lysis, and
a second container comprising a second set of reagents for performing a reverse transcription and loop-mediated isothermal nucleic acid amplification reaction (RT-LAMP),
wherein the second set of reagents comprises a first set of oligonucleotide primers directed to a first target nucleic acid of the SARS-CoV-2 virus selected from the group consisting of Set 1, Set 2, Set 3, and Set 4, identified by SEQ ID NOs 1-6 (Set 1), SEQ ID NOs 7-12 (Set 2), SEQ ID NOs 7, 9, 11, 13-15 (Set 3), and SEQ ID NOs 7-12, 16, 17 (Set 4); and a second set of oligonucleotide primers directed to a second target nucleic acid of the human subject, optionally wherein the second target nucleic acid is a polynucleotide of a beta-actin RNA molecule.

30. The kit of claim 29, wherein the first set of reagents each comprises an RNase inhibitor.

31. The kit of claim 29 or 30, wherein the second set of reagents further comprises an RNase inhibitor, deoxyribonucleotide triphosphates (dNTPs) including dUTP, a source of magnesium ions, a reverse transcriptase, and a DNA polymerase.

32. The kit of any one of claims 29-31, wherein the second set of reagents further comprises a fluorescent DNA intercalating agent and a set of detection primers adapted to contain a quencher-fluorophore duplex region, wherein the set of detection primers is directed to either the first or second target nucleic acid.

33. The kit of any one of claims 29-32, wherein the second set of reagents further comprises one or more of an RNase inhibitor, a serum albumin, a reducing agent such as tris(2-carboxyethyl)phosphine (TCEP) and salts thereof, and thermolabile uracil-DNA glycosylase (UDG).

34. The kit of any one of claims 29-33, wherein the second set of reagents is lyophilized.

35. The kit of claim 34, wherein the second set of reagents further comprises one or more of dextran, mannitol, sorbitol, maltodextrin, trehalose, lactose, and/or lactitol.
